(19) United States
(12) Patent Application Publication
(10) Pub. No.: US 2021/0030829 A1
(43) Pub. Date: Feb. 4, 2021

(54) METHODS FOR TREATMENT OF CANCER AND ENHANCEMENT OF NANOPARTICLE ACCUMULATION IN TISSUES

(71) Applicant: University of Louisville Research Foundation, Inc., Louisville, KY (US)

(72) Inventor: Huang-Ge Zhang, Louisville, KY (US)

(73) Assignee: University of Louisville Research Foundation, Inc., Louisville, KY (US)

(21) Appl. No.: 16/978,615

(22) PCT Filed: Mar. 6, 2019
(86) PCT No.: PCT/US19/20971

§ 371 Date: Sep. 4, 2020

Related U.S. Application Data
(60) Provisional application No. 62/639,300, filed on Mar. 6, 2018.

(51) Int. Cl. A61K 36/886; A61K 9/00; A61P 35/04; A61K 9/127; A61K 9/16

(52) U.S. Cl. CPC A61K 36/886; A61K 9/0019; A61K 9/16; A61K 9/127; A61P 35/04

(57) ABSTRACT

Provided are methods for treating tumors and/or cancers. In some embodiments, the methods relate to administering to a subject in need thereof an effective amount of a nanoparticle derived from an edible plant and an effective amount of an autologous exosome. Also provided are methods for enhancing accumulation of nanoparticles in the lungs of subjects, methods for delivering agents to the liver, brain, and/or bones of subjects, and methods for delivering agents across the blood-brain barrier of subjects.

### CROSS REFERENCE TO RELATED APPLICATION

This application claims the benefit of U.S. Provisional Patent Application Ser. No. 62/639,300 filed Mar. 6, 2018, the disclosure of which is incorporated herein by reference in its entirety.

### GOVERNMENT INTEREST

This invention was made with government support under grant numbers TR000875 and R01 AT008617 awarded by the National Institutes of Health. The government has certain rights in the invention.

### TECHNICAL FIELD

The presently disclosed subject matter generally relates to methods for treatment of cancer and enhancement of nanoparticle accumulation in tissues. In particular, some embodiments of the presently disclosed subject matter relate to methods for treating cancer and enhancing nanoparticle accumulation in a tissue where an effective amount of autologous exosomes and an effective amount of the nanoparticles are administered to the subject. Also provided are methods and compositions for delivering active agents across the blood-brain barrier in subjects.

### BACKGROUND

Despite many potential advantages for therapeutic delivery, a nanoparticle-based delivery system has to overcome many hurdles such as eliminating the induction of cytotoxic effects due to off targeting. Unlike the situation with nanoparticles synthesized artificially, nano-sized exosome-like nanoparticles from edible plants have been utilized for encapsulating drugs, siRNA, DNA expression vectors, and antibodies to treat diseases in mouse models without causing side effects. Although the use of edible plant-derived exosome-like nanovectors in therapeutic delivery holds great promise, effective and efficient delivery of agents to desired targets remains challenging.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter provides methods for treating tumors and/or cancers. In some embodiments, the methods comprise administering to a subject in need thereof an effective amount of a nanoparticle derived from an edible plant and an effective amount of an autologous exosome. In some embodiments, the nanoparticle derived from an edible plant comprises, optionally encapsulates, an effective amount of a therapeutic agent. In some embodiments, the therapeutic agent is a chemotherapeutic agent. In some embodiments, the autologous exosome is administered at least 30 minutes prior to the administration of a nanoparticle derived from the edible plant. In some embodiments, the cancer is lung cancer. In some embodiments, the lung cancer is a metastasis in the lung. In some embodiments, the metastasis is secondary to a melanoma or a breast cancer.

The presently disclosed subject matter also provides in some embodiments methods for enhancing accumulation of nanoparticles in the lungs of subjects. In some embodiments, the methods comprise administering an effective amount of an autologous exosome to the subject and administrating an effective amount of the nanoparticle to the subject subsequent to the administration of the autologous exosome. In some embodiments, the nanoparticle comprises, optionally encapsulates, an effective amount of a therapeutic agent. In some embodiments, the nanoparticle is derived from an edible plant.

In some embodiments, the presently disclosed subject matter also provides in some embodiments methods for delivering agents to the liver, brain, and/or bones of subjects in need thereof. In some embodiments, the methods comprise administering to the subject an effective amount of an aloe-derived exosome-like nanoparticle (AELN) comprising, optionally encapsulating, the agent, wherein the administering is via a route of administration such that AELN enters the subject's circulation. In some embodiments, the agent is a therapeutic agent, optionally a chemotherapeutic agent. In some embodiments, the subject has a disease, disorder, or condition of the liver, brain, and/or bone at least one symptom and/or consequence of which can be ameliorated by the agent. In some embodiments, the route of administration is intravenous administration.

The presently disclosed subject matter also provides in some embodiments methods for delivering agents across the blood-brain barrier of subjects. In some embodiments, the methods comprise administering to a subject an effective amount of an aloe-derived exosome-like nanoparticle (AELN) comprising, optionally encapsulating, an agent via a route of administration wherein the AELN enters the subject's circulation, thereby resulting in the AELN contacting the blood-brain barrier of the subject, whereby the agent is delivered across the blood-brain barrier of the subject. In some embodiments, the agent is a therapeutic agent, optionally a chemotherapeutic agent. In some embodiments, the subject has a disease, disorder, or condition of the brain at least one symptom and/or consequence of which is treatable with the agent.

Thus, it is an object of the presently disclosed subject matter to provide compositions and methods for treatment of cancer and enhancement of nanoparticle accumulation in tissues.

An object of the presently disclosed subject matter having been stated above, other objects and advantages will become apparent upon a review of the following Detailed Description and EXAMPLES, particularly in view of the Figures.

### BRIEF DESCRIPTION OF THE DRAWINGS

FIGS. 1A-1C present the results of experiments showing distribution of nanovectors in mice. Nanovectors, including grapefruit-derived nanovectors (GNVs; #1), lymphocyte membrane-coated GNVs, IGNVs (#2), DOTAP:DOPE liposomes (#3), and liposomes from Avanti Polar Lipids (#4), were labeled with DiR dye (Sigma-Aldrich Corporation, St. Louis, Mo., United States of America) and injected intravenously into normal mice. FIG. 1A is a series of representative live body images of DIR-labeled nanovectors in mice (FIG. 1A) collected at different time points (30 minutes, 60 minutes, 6 hours, and 12 hours). NC: negative control. Mice were then sacrificed, and organs including liver, spleen, lung, kidney, heart, thymus, brain, and stomach were removed. DiR signals in organs were detected (FIG. 1B, left panel) and quantified (FIG. 1B, right panel; in each case, liver>spleen>lung>stomach, except for #1, in which stomach was>lung) by scanning using a KODAK Imaging Station 4000 mm Pro. FIG. 1C shows representative images of cell targets of PKH26-labeled nanovectors in liver. Nanovectors were labeled with PKH26 and injected intravenously into mice. Livers from mice were removed and tissue sections were stained with an anti-mouse F4/80 antibody. DAPI: 4′,6-diamidino-2-phenylindole nuclear stain. Particles: PKH26-labeled nanovectors. F4/80: anti-F4/80-antibody stained tissue sections. Merge: overlays of DAPI, Particles, and F4/80 panels.

FIG. 2 is a series of fluorescence micrographs showing Kupffer cell depletion by clodrosomes. Mice were injected intravenously with clodrosomes (700 μg in 150 μl) and Kupffer cells were stained with anti-mouse F4/80 antibody in mouse liver tissue sections 24, 48, and 72 hours post-injection. Representative images of antibody F4/80-stained liver tissue are presented.

FIGS. 3A-3C depict the results of experiments showing biodistribution of GNV nanovectors after Kupffer cell depletion. 24 hours after treatment with clodrosomes, mice were injected intravenously with DiR dye-labeled GNVs (200 nmol; FIG. 3A, left panel) and live images were obtained at different time points (30 minutes, 60 minutes, and 180 minutes; FIG. 3A, middle panel). A representative image from each group of mice is shown. The right panel of FIG. 3A is a graph of a quantification of fluorescence intensity presented as the mean net intensity (Sum Intensity/Area; n=5), Data are presented as mean±SD. ***p<0.001 normal, untreated mice (circles) versus Kupffer cell depleted mice (squares). Error bars represent SD. For statistics, see the section entitled “Statistical analysis” herein below. Organs including liver, lung, spleen, kidney, brain, thymus, heart, and stomach were also isolated and scanned. Representative images of each organ are presented in FIG. 3B, left panel. A graphical representation presented as the mean net intensity (Sum Intensity/Area; n=5) is presented in FIG. 3B, right panel. ***p<0.001 normal (untreated) mice (circles) versus Kupffer cell depleted mice (squares). PKH26-labeled GNVs were injected intravenously into Kupffer cell depleted-mice and colocalization of PKH-126 GNVs with F4/80′ Kupffer cells was examined using confocal microscopy. Representative images of anti-F4/80 stained tissues are presented in FIG. 3C. DAPI: 4′,6-diamidino-2-phenylindole nuclear stain. Particles: PKH26-labeled nanovectors. F4/80: anti-F4/80-antibody stained tissue sections. Merge: overlays of DAPI, Particles, and F4/80 panels.

FIGS. 4A-4I presented the results of experiments showing that the majority of circulating exosomes were taken up by liver F4/80 macrophages, and pre-injection of exosomes led to redirecting subsequently injected nanovectors from the liver to the lungs. Exosomes from normal mouse plasma were isolated using the PUREEXO® brand Exosomes Isolation kit (101BIO, Mountain View, Calif., United States of America). The morphologies of exosomes were examined and imaged using transmission electron microscopy (TEM; FIG. 4A). Size distributions (FIG. 4B) and surface Zeta potentials (FIG. 4C) of exosomes were measured using a ZetaSizer (Malvern Panalytical Ltd., Westborough, Mass., United States. In FIG. 4D, the distribution of 1,1-dioctadecyl-3,3,3′3′-tetramethylindotricarbocyanine-iodide (DiR) labeled exosomes in normal mice is shown. Mice were injected intravenously with 200 μg of exosomes and DiR signals in the liver, lung, spleen, kidney, heart, thymus, brain and stomach was analyzed by scanning using KODAK Imaging Station 4000 mm Pro (KODAK Carestream Health, Rochester, N.Y., United States of America; FIG. 4D, left panel) and quantified (FIG. 4D, right panel) in the liver (82.3%), lung (3.7%), and spleen (13.9%). Livers from mice were removed over a 24- to 72-hour period after intravenous (i.v.) injection and liver tissue sections were stained with a rat anti-F4/80 antibody (Abcam, Cambridge, Mass., United States of America). Representative images of DiR-labeled exosomes from mice and F4/80 stained liver section are shown in FIG. 4E. The bar in each panel of FIG. 4E is 50 μm. For FIG. 4F, exosomes were isolated from plasma of normal mice and injected intravenously into mice. DiR-labeled nanovectors including grapefruit lipid-derived GN (#1), lymphocyte membrane-coated GNVs-IGNVs (#2), DOTAP:DOPE liposomes (#3) or liposomes from Avanti Polar Lipids, Inc. (#4; Alabaster, Ala., United States of America) and injected intravenously into mice 30 minutes after an injection of exosomes (see the schematic at the top of FIG. 4F). Accumulation of nanovectors in mouse liver was examined in living mice at 10, 30, and 720 minutes post-injection (FIG. 4F, left panel) and in livers (FIG. 4F, top right panel). Representative images of DiR-labeled nanovectors from mice (FIG. 4F, left panel) and livers (FIG. 4F, top right panel) are presented. The middle panel of FIG. 4F is a series of graphical representations of the intensities in the left panel of FIG. 4F presented as the mean net intensity (Sum Intensity/Area; n=5). Data are presented as mean±standard deviation (SD). *p<0.05; **p<0.01; ***p<0.001 of untreated (black squares) vs. exosome-treated (Exo-block; gray squares). These data are also presented in the bar graph in the bottom of the right panel. Error bars represent SD. For statistics, see the section below entitled “Statistical analysis”. For FIG. 4G, PKH67-labeled GNVs (200 nmol) were injected intravenously into mice pre-i.v. injected with exosomes. Liver, lung, and spleen were removed and PKH67-GNVs in tissues sections stained with anti-F4/80 antibody were imaged using confocal microscopy. Representative images are shown. Inhibition of liver accumulation of nanovectors occurred in a dose-dependent manner. Different doses of C57BL/6 mice serum-derived exosomes (25, 50, 100, and 200 μg) were injected intravenously into C57BL/6 mice. 30 minutes after injection of exosomes, mice were injected with 200 nmol DiR dye-labeled GNVs. DiR dye signals in living mice (FIG. 4H, left panel) were quantitatively analyzed using a KODAK Imaging Station 4000 mm Pro. The data are presented as the mean net intensity (Sum Intensity/Area; n=5) in the tight panel of FIG. 4H. Data are presented as mean±SD. **p<0.01; ***p<0.001. Error bars represent SD. Six-week-old B6 mice (n=5) were injected i.v. with autologous blood-derived exosomes (200 μg in 100 μL PBS) or PBS alone as a control. Body weight was measured over the period of 3 weeks and expressed as percentage of gained body weight over the 3-week period in FIG. 4I. NS: not significant.

FIGS. 5A-5D present the results of experiments showing that exosomes redirected nanovectors from liver to the lungs and the tumor. DiR dye-labeled. GNVs were injected intravenously into 6-week old female BALB/c mice pretreated with exosomes or clodrosomes to deplete Kupffer cells, or PBS as a control (Normal). A negative control (NC) was also tested. Anticoagulant peripheral blood was collected 30, 60, and 180 minutes after injection. The DiR dye signals in blood were assayed (FIG. 5A, left panel) and quantified (FIG. 5A, right panel) by scanning using KODAK Imaging Station 4000 mm Pro. The data in FIG. 5A, right panel are presented as the mean net intensity (Sum Intensity/Area; n=5). Data are presented as mean±SD, ***p<0.001. Error bars represent SD. DiR dye-labeled GNVs were injected intravenously into 4T1 bearing mice pretreated with exosomes or PBS as a control. 4T1 tumor bearing mice without any treatment were used as a negative control (NC). Representative whole-body images collected at 1 hour, 3 hours, 6 hours, and 20 hours after injection are presented in the left panel of FIG. 5B. The data are presented as the mean net intensity (Sum Intensity/Area; n=5) in the right panel of FIG. 5B. NC: circles. GNVs: squares. GNVs plus exosomes (GNVs/Exo block): triangles. Data are presented as mean±SD. **p<0.01; ***p<0.001. Error bars represent SD. For FIG. 5C, Mice were sacrificed and tumors and organs (liver, lung, spleen, heart, thymus, kidney, and lymph node) were isolated, scanned using a KODAK Imaging Station 4000 mm Pro (FIG. 5C, left panel), and quantitatively analyzed. The data are presented as the mean net intensity (Sum Intensity/Area; n=5) in FIG. 5C, right panel. Data are presented as mean SD. *p<0.05; **p<0.01; ***p<0.001. Error bars represent SD. For FIG. 5D, the distribution of paclitaxel in B16F10 tumor-bearing mice was determined. B16F10 tumor-bearing mice were injected intravenously with paclitaxel-loaded GNV (GNV-PTX) 3 times. The concentrations of paclitaxel in mouse tumor, liver, lung, and spleen were analyzed by HPLC (n=3). Data are presented as mean±t=SD. p<0.05; **p<0.01; ***p<0.001. Error bars represent SD. For each of Free PTX, GNV-PTX, and Exo/GNV-PTX, the bars from left to right are liver, lung, spleen, and tumor.

FIGS. 6A and 6B are data from spectrophotometric analyses of loading efficiencies of doxorubicin and paditaxel on GNVs. GNV-Dox and GNV-PTX were prepared by bath-sonication, the residual Dox (FIG. 6A) or PTX (FIG. 6B) in the supernatant was quantitatively analyzed by UV-Visible spectrophotometer at 486 and 265 nm, respectively, and the loading efficiency was calculated and expressed as (Total drug—amount of drug in the supernatant)/Total drug×100% in FIG. 6C. Error bars represent SD.

FIGS. 7A-7F presented the results of experiments showing that pre-injection of blood-derived exosomes enhanced anti-tumor metastasis of therapeutic agents delivered by GNVs. 1×10⁵ 4T1 cells were injected at a mammary fat pad of female BALB/c mice. Beginning on day 5 after the injection, mice were tail vein-injected every 3 days for a total of 10 times with PBS, GNV-Dox, Exo/GNV-Dox, GNV-miR18a, Exo/GNV-miR18a, GNV-miR18a/Dox, or Exo/GNV-miR18a/Dox. Mice were then sacrificed, and lungs were imaged (FIG. 7A, left panel) and the number of pulmonary metastatic nodules were quantified (bar graph, FIG. 7A, right panel). Lung tissue sections were also stained with H&E (FIG. 7B). Representative images of lung and sectioned lung tissue (n=5), and survival rates of mice were recorded (FIG. 7C; left-most trace: PBS. Next adjacent trace: Dox. Next adjacent trace: GNV-Dox. Right-most trace: Exo/GNV-Dox). B16F10 cells (5×10⁴) were injected i.v. into C57BL/6 mice. Beginning 5 days later, mice were tail vein injected every 3 days for a total of 10 with PBS, PTX, GNV-PTX, or Exo/GNV-PTX. Lungs were removed, imaged (FIG. 7D, left panel) and the metastatic nodules in lungs were quantitative analyzed (FIG. 7D, right panel), Tissue sections were also stained with H&E (FIG. 7E), and survival rates of mice were recorded (FIG. 7F; left-most trace: PBS. Next adjacent trace: PTX. Next adjacent trace: GNV-PTX. Right-most trace: Exo/GNV-PTX). Data are presented as mean±SD. *p<0.05; **p<0.01, and ***p<0.001. Error bars represent SD. The data shown in FIG. 7 were representative of at least 3 independent experiments (n=5).

FIGS. 8A-8C presented the results of experiments showing that pre-injection of exosomes prevented co-localization of CD36 and GNVs, and knockout of CD36 led to cancellation of exosome-mediated inhibition of liver uptake of GNVs. Six-week old B6 mice were injected intravenously with DiR-GNVs (2 nMol/100 μL). 30 minutes after injection, mice were sacrificed, and liver sections were immuno-stained with anti-CD36 and anti-F4/80 antibodies. Representative images of sectioned liver tissue (FIG. 8A; n=5) stained with anti-CD36 (CD36) and anti-F4/80 (F4/80) antibodies are shown. Six-week old B6 mice were injected intravenously with exosomes (3×10¹⁰ exosomes/100 μL) isolated from peripheral blood or PBS as a control, 30 minutes after the injection, DiR-GNVs (2 nMol/100 μL) were administered intravenously. The mice were sacrificed and F4/80 positive cells were FACS sorted and stained with an anti-CD36 antibody. Representative confocal images of F4/80 positive cells isolated from liver are shown (FIG. 8B; n=5). Wild type B6 mice and age/sex-matched CD36 KO mice were injected intravenously with to C57BJ/6 plasma exosomes (3×10¹⁰ exosomes/mouse, n=5) followed by DiR-labeled GNVs (700 nMol) at 30-minute intervals. The DiR signal in living mice (FIG. 8C, top panel) and liver tissue (FIG. 8C, bottom panel) were imaged 30 minutes after the GNV injection.

FIGS. 9A and 9B present the results of experiments showing that siRNA knockdown of IGFR1 reversed exosome-mediated inhibition of GNVs uptake by human monocytes. U937 human monocytes cells were incubated for 30 minutes with exosomes (3×10⁸ nanovectors) isolated from healthy subjects. Treated cells were then incubated with PKH26-labeled GNVs (2 nMol) for additional 0, 30, 60, 90, or 120 minutes, and the cells were subsequently FACS analyzed. Representative FACS images of GNV positive cells (n=5) are presented in FIG. 9A. 48-hour siRNA-transfected U937 human monocytes cells were incubated with/without exosomes for 30 minutes. PKH126-labeled GNVs were added to the treated cells and incubated for additional 60 minutes before cells were harvested for FACS analysis of PKH26 positive cells. Representative FACS images of GNV positive cells are presented in FIG. 9B, left panel, and the percentages of GNVs⁺U937 cells as a result of siRNA IGFR1 knockdown are represented as mean±SD in FIG. 9B, right panel. *p<0.05. Error bars represent SD. siRNA knockdown of LTK, IGFR1, and FYN (circled) all had an effect on uptake of exosomes.

FIG. 10 is a series of representative infrared-scanned images of liver, spleen, lung, brain, bone, and kidney isolated from male C57BL/6 mice administered DiR dye-labeled aloe ELN (Aloe ELNs^{Dir}, 50 mg per mouse in 100 μl PBS) by intravenously injection imaged at day 10 after administration. Representative images using an Odyssey Infrared Imager (LI-COR Inc., Lincoln, Nebr., United States of America) are presented. Results represent one of three independent experiments. Aloe exosome-like nanovectors (AELNs) preferentially homed to brain and bone.

### DETAILED DESCRIPTION

The details of one or more exemplary embodiments of the presently disclosed subject matter are set forth herein. Modifications to the exemplary embodiments described herein, and other representative embodiments, will be evident to those of ordinary skill in the art after a study of the information provided herein. The information provided herein, and particularly the specific details of the described exemplary embodiments, is provided primarily for clearness of understanding and no unnecessary limitations are to be understood therefrom. In case of conflict, the present disclosure, including definitions, will control.

While the terms used herein are believed to be well understood by those of ordinary skill in the art, certain definitions are set forth to facilitate explanation of the presently disclosed subject matter.

Unless defined otherwise, all technical and scientific terms used herein have the same meaning as is commonly understood by one of skill in the art to which the invention(s) belong.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids, and other compounds, are, unless indicated otherwise, in accord with their common usage, recognized abbreviations, or the IUPAC-IUB Commission on Biochemical Nomenclature (see IUPAC-IUB Commission, 1972).

Although any methods, devices, and materials similar or equivalent to those described herein can be used in the practice or testing of the presently disclosed subject matter, representative methods, devices, and materials are described herein.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used herein, including in the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently disclosed subject matter relates in some embodiments to methods for treatment of cancer and enhancement of nanoparticle accumulation in a tissue. In particular, some embodiments of the presently disclosed subject matter include methods for treating cancer and enhancing nanoparticle accumulation in a tissue where an effective amount of autologous exosomes and an effective amount of the nanoparticles are administered to the subject.

In some embodiments of the presently disclosed subject matter, a method of treating a cancer is provided that comprises administering to a subject an effective amount of a nanoparticle derived from an edible plant and an effective amount of an autologous exosome. In some embodiments, the nanoparticle derived from an edible plant encapsulates an effective amount of a therapeutic agent.

The term “nanoparticle” as used herein in reference to the edible plant-derived nanoparticles of the presently disclosed subject matter, refers to nanoparticles that are in the form of small assemblies of lipid particles, are about 50 to 1000 nm in size, and are not only secreted by many types of in vitro cell cultures and in vivo cells, but are also commonly found in vivo in body fluids, such as blood, urine and malignant ascites. Indeed, such nanoparticles include, but are not limited to, particles such as microvesicles, exosomes, nanovesicles, nanovectors, epididimosomes, argosomes, exosome-like vesicles, microparticles, promininosomes, prostasomes, dexosomes, texosomes, dex, tex, archeosomes, and oncosomes.

Such nanoparticles can be formed by a variety of processes, including the release of apoptotic bodies, the budding of microvesicles directly from the cytoplasmic membrane of a cell, and exocytosis from multivesicular bodies. For example, exosomes are commonly formed by their secretion from the endosomal membrane compartments of cells as a consequence of the fusion of multivesicular bodies with the plasma membrane. The multivesicular bodies are formed by inward budding from the endosomal membrane and subsequent pinching off of small vesicles into the luminal space. The internal vesicles present in the multivesicular bodies are then released into the extracellular fluid as so-called exosomes.

As part of the formation and release of nanoparticles, unwanted molecules are eliminated from cells. However, cytosolic and plasma membrane proteins are also incorporated during these processes into the microvesicles, resulting in microvesicles having particle size properties, lipid bilayer functional properties, and other unique functional properties that allow the nanoparticles to potentially function as effective nanoparticle carriers of therapeutic agents. In this regard, in some embodiments, the term “nanoparticle” is used interchangeably herein with the terms “microvesicle,” “liposome,” “exosome,” “exosome-like particle,” “nanovector” and grammatical variations of each of the foregoing.

The term “edible plant” is used herein to describe organisms from the kingdom Plantae that are capable of producing their own food, at least in part, from inorganic matter through photosynthesis, and that are fit for consumption by a subject, as defined herein below. Such edible plants include, but are not limited to, vegetables, fruits, nuts, and the like. In some embodiments of the nanoparticle compositions described herein, the edible plant is a fruit, In some embodiments, the fruit is selected from a grape, a grapefruit, and a tomato. In some embodiments, the edible plant is selected from a ginger, a grapefruit, and a carrot. In some embodiments, the edible plant is ginger,

The phrase “derived from an edible plant,” when used in the context of a nanoparticle derived from an edible plant, refers to a nanoparticle that, by the hand of man, exists apart from its native environment and is therefore not a product of nature. In this regard, in some embodiments, the phrase “derived from an edible plant” can be used interchangeably with the phrase “isolated from an edible plant” to describe a nanoparticle of the presently disclosed subject matter that is useful for encapsulating therapeutic agents.

The phrase “encapsulated by a nanoparticle,” or grammatical variations thereof is used herein to refer to nanoparticles whose lipid bilayer surrounds a therapeutic agent. For example, a reference to “nanoparticle chemotherapeutic agent” refers to a nanoparticle whose lipid bilayer encapsulates or surrounds an effective amount of a chemotherapeutic agent. In some embodiments, the encapsulation of various therapeutic agents within nanoparticles can be achieved by first mixing one or more therapeutic agents with isolated nanoparticles in a suitable buffered solution, such as phosphate-buffered saline (PBS). After a period of incubation sufficient to allow the therapeutic agent to become encapsulated during the incubation period, the nanoparticle/therapeutic agent mixture is then subjected to a sucrose gradient (e.g., and 8, 30, 45, and 60% sucrose gradient) to separate the free therapeutic agent and free microvesicles from the therapeutic agents encapsulated within the microvesicles, and a centrifugation step to isolate the nanoparticles encapsulating the therapeutic agents. After this centrifugation step, the nanoparticles including the therapeutic agents are seen as a band in the sucrose gradient such that they can then be collected, washed, and dissolved in a suitable solution for use as described herein below.

In some embodiments of the presently disclosed subject matter, the therapeutic agent encapsulated by the nanoparticle is a chemotherapeutic agent. Examples of chemotherapeutic agents that can be used in accordance with the presently disclosed subject matter include, but are not limited to, platinum coordination compounds such as cisplatin, carboplatin or oxalyplatin; taxane compounds, such as paclitaxel or docetaxel; topoisomerase I inhibitors such as camptothecin compounds for example irinotecan or topotecan; topoisomerase II inhibitors such as anti-tumor podophyllotoxin derivatives for example etoposide or teniposide; anti-tumor vinca alkaloids for example vinblastine, vincristine or vinorelbine; anti-tumor nucleoside derivatives for example 5-fluorouracil, gemcitabine or capecitabine; alkylating agents, such as nitrogen mustard or nitrosourea for example cyclophosphamide, chlorambucil, carmustine or lomustine; anti-tumor anthracycline derivatives for example daunorubicin, doxorubicin, idarubicin or mitoxantrone; HER2 antibodies for example trastuzumab; estrogen receptor antagonists or selective estrogen receptor modulators for example tatnoxifen, toremifene, droloxifene, faslodex or raloxifene; aromatase inhibitors, such as exemestane, anastrozole, letrazole and vorozole; differentiating agents such as retinoids, vitamin D and retinoic acid metabolism blocking agents (RAMBA) for example accutane; DNA methyl transferase inhibitors for example azacytidine; kinase inhibitors for example flavoperidol, imatinib mesylate or gefitinib; farnesyltransferase inhibitors; HDAC inhibitors; other inhibitors of the ubiquitin-proteasome pathway for example VELCADE® (Millennium Pharmaceuticals, Cambridge, Mass.); or YONDELIS® (Johnson & Johnson, New Brunswick, N.J.). In some embodiments, the chemotherapeutic agent that is encapsulated by an exosome in accordance with the presently disclosed subject matter is selected from retinoic acid, 5-fluorouracil, vincristine, actinomycin D, adriamycin, cisplatin, docetaxel, doxorubicin, and taxol.

As used herein, the term “cancer” refers to all types of cancer or neoplasm or malignant tumors found in animals, including leukemias, carcinomas, melanoma, and sarcomas. By “leukemia” is meant broadly progressive, malignant diseases of the blood-forming organs and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

The term “carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “sarcoma” generally refers to a tumor which is made up of a substance like the embryonic connective tissue and is generally composed of closely packed cells embedded in a fibrillar or homogeneous substance. Sarcomas include, for example, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma subungal melanoma, and superficial spreading melanoma.

Additional cancers include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer. In some embodiments, the cancer is lung cancer. In some embodiments, the lung cancer is a metastasis in the lung, which is, in certain embodiments, secondary to a melanoma or a breast cancer.

As used herein, the terms “treatment” or “treating” relate to any treatment of a condition of interest (e.g., an inflammatory disorder or a cancer), including but not limited to prophylactic treatment and therapeutic treatment. As such, the terms “treatment” or “treating” include, but are not limited to: preventing a condition of interest or the development of a condition of interest; inhibiting the progression of a condition of interest; arresting or preventing the further development of a condition of interest; reducing the severity of a condition of interest; ameliorating or relieving symptoms associated with a condition of interest; and causing a regression of a condition of interest or one or more of the symptoms associated with a condition of interest.

For administration of a therapeutic composition as disclosed herein (e.g., an edible plant-derived nanoparticle encapsulating a chemotherapeutic agent), conventional methods of extrapolating human dosage based on doses administered to a murine animal model can be carried out using the conversion factor for converting the mouse dosage to human dosage: Dose Human per kg=Dose Mouse per kg/12 (Freireich et al., 1966). Doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich et al., 1966). Briefly, to express a mg/kg dose in any given species as the equivalent mg/m² dose, multiply the dose by the appropriate km factor. In an adult human, 100 mg/kg is equivalent to 100 mg/kg×37 kg/sq m=3700 mg/m².

Suitable methods for administering a therapeutic composition in accordance with the methods of the presently disclosed subject matter include, but are not limited to, systemic administration, parenteral administration (including intravascular, intramuscular, and/or intraarterial administration), oral delivery, buccal delivery, rectal delivery, subcutaneous administration, intraperitoneal administration, inhalation, intratracheal installation, surgical implantation, transdermal delivery, local injection, intranasal delivery, and hyper-velocity injection/bombardment. Where applicable, continuous infusion can enhance drug accumulation at a target site (see, e.g., U.S. Pat. No. 6,180,082). In some embodiments, the autologous exosome is administered prior to the administration of a nanoparticle derived from the edible plant.

Regardless of the route of administration, the compositions of the presently disclosed subject matter are typically administered in amount effective to achieve the desired response. As such, the term “effective amount” is used herein to refer to an amount of the therapeutic composition (e.g., a nanoparticle encapsulating a therapeutic agent, and a pharmaceutically vehicle, carrier, or excipient) sufficient to produce a measurable biological response (e.g., a decrease in cancer cells). Actual dosage levels of active ingredients in a therapeutic composition of the present invention can be varied so as to administer an amount of the active compound(s) that is effective to achieve the desired therapeutic response for a particular subject and/or application. Of course, the effective amount in any particular case will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

For additional guidance regarding formulation and dose, see U.S. Pat. Nos. 5,326,902; 5,234,933; PCT International Patent Application Publication No. WO 93/25521; Berkow et al., 1997; Goodman et 1996; Ebadi 1998; Katzung 2001; Remington et al., 1975; Speight et al., 1997; Duch et al., 1998.

Still further provided, in some embodiments, are methods of enhancing accumulation of a nanoparticle in a lung of a subject, that comprise administering an effective amount of an autologous exosome to the subject and administrating an effective amount of the nanoparticle derived from an edible plant to the subject subsequent to the administration of the autologous exosome.

As used herein, the term “subject” includes both human and animal subjects. Thus, veterinary therapeutic uses are provided in accordance with the presently disclosed subject matter. As such, the presently disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

The practice of the presently disclosed subject matter can employ, unless otherwise indicated, conventional techniques of cell biology, cell culture, molecular biology, transgenic biology, microbiology, recombinant DNA, and immunology, which are within the skill of the art. Such techniques are explained fully in the literature. See e.g., Sambrook et al., 1989; U.S. Pat. No. 4,683,195; Glover 1985; Gait 1984; Hames & Higgins, 1985; Hames & Higgins, 1984; Freshney 2016; Woodward 1985; Perbal 1984; Miller & Calos, 1987; Wu & Grossman, 1987; Mayer & Walker, 1987; Herzenberg et al., 1996.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

### Introduction to the Examples

The following EXAMPLES relate to an investigation that liver macrophages are the primary cells that take the exosomes out of the peripheral blood. The liver is the major site for removing circulating macromolecules including nano-sized exosome-like nanoparticles such as grapefruit exosome-like nanoparticles made from grapefruit-derived lipids. The rapid sequestration of intravenously injected nanovectors from the blood by Kupffer cells is one of major challenges for efficient delivery of targeted drug carriers to a desired cell population and for prevention of liver toxicity.

### Materials and Methods for the Examples

Mice. C57BL/6j, BALB/c, and CD36 knockout mice, 6-8 weeks of age were obtained from The Jackson Laboratory, Bar Harbor, Me., United States of America. All animal procedures were approved by the Institutional Animal Care and Use Committee of the University of Louisville (Louisville, Ky., United States of America).

Reagents and antibodies. Mouse monoclonal anti-CD36 and rat anti-F4/80 were purchased from ABcam (Cambridge, Mass., United States of America). Primary antibodies were detected by ALEXAFLUOR® 488-conjugated, ALEXAFLUOR® 594-conjugated, or ALEXAFLUOR® 647-conjugated goat anti-mouse, anti-rabbit IgG, and anti-rat (1:600, Invitrogen Corp. Carlsbad, Calif., United States of America). Tissues were counterstained with DAPI and images were captured on a Zeiss LSM 510 confocal microscope equipped with a digital image analysis system (Pixera Corporation, San Jose, Calif., United States of America).

Near-infrared lipophilic carbocyanine dye 1,1-dioctadecyl-3,3,3′,3′-tetramethylindotricarbocyanine-iodide (DIR) was purchased from Invitrogen Corp. (Carlsbad, Calif., United States of America), PKH26-GL and PKH67 (Sigma-Aldrich, St. Louis, Mo., United States of America), PUREEXO® Exosome Isolation Kit for serum (101BIO, Palo Alto, Calif., United States of America) and clodrosomes (Encapsula NanoSciences LLC, Brentwood, Tenn., United States of America) were purchased. Human ON-TARGETplus—Tyrosine Kinase—SMARTpool black plates (version 2.0, Dharmacon, Lafayette, Colo., United States of America) were purchased ready to use at a final concentration 50 nM.

The murine melanoma cell line B16F10, the murine breast tumor cell line 4T1, and human U937 monocytes were purchased (American Type Culture Collection (ATCC), Manassas, Va., United States of America), and cultured according to the supplier's instructions.

In vivo imaging. To check distribution of particles in mice, DIR dye-labeled particles including GNVs, IGNVs, DOTAP:DOPE (1:1 w/w), and liposomes from Avanti Polar Lipids (Alabaster, Ala., United States of America) were prepared as follows. 200 nmol of grapefruit lipids, DOTAP:DOPE (1:1, w/w), were dried in glass vials and DIR dye was added with ddH₂O. The particles were prepared according to the protocol described in Wang et al. (2013) Delivery of therapeutic agents by nanoparticles made of grapefruit-derived lipids Nature Communications 4:1867 (see also U.S. Patent Application Publication Nos. 2016/0045448 and 2017/0035700, both of which are incorporated by reference in their entireties). Free DIR dye was removed by centrifugation at 100,000 g for 1 hour. The DiR dye-labeled particles were injected into mice via the tail vein and images of living mice were obtained 0.5, 1, 6, and 12 hours after injection. DiR dye signals in organs were quantified by scanning mice using a KODAK Imaging Station 4000 mm Pro.

To verify the distribution of exosomes, exosomes (200 μg) isolated from plasma of normal BALB/c or C57BL/6j mice were labeled with DiR dye and injected into mice. Organs were removed and DiR dye signals in each organ were quantified 12 hours after injection.

To demonstrate the redistribution of particles in mice treated with clodrosomes (700 μg) or exosomes (200 μg), DiR dye-labeled GNVs were injected intravenously into mice (24 hours after clodrosome injection and 1 hour after exosomes injection), respectively. DiR dye signals in living mice and organs were quantified using a KODAK Imaging Station 4000 mm Pro.

To examine the redistribution of particles in 4T1 tumor-bearing mice, 200 μg of mouse blood-derived exosomes or DiR. dye-labeled GNVs were intravenously injected into mice and DiR signals in living mice, 4T1 tumor tissue, liver, lung, spleen, kidney, thymus, heart, and lymph node were analyzed.

Monitoring GNVs in peripheral blood. DiR dye-labeled GNVs (200 nmol) were injected intravenously into mice receiving clodrosome or exosome treatment. Next, 100 μl of anticoagulated blood was collected at different time points (30, 60, and 180 minutes) and the DiR signals were quantified using a KODAK Imaging Station 4000 mm Pro to scan the samples.

Exosomes isolation. Exosomes from mouse plasma were isolated according to the manual of the PUREEXO® brand Exosomes Isolation kit (101BIO). In brief, debris in plasma was removed by centrifugation at 2000×g for 10 minutes. The supernatant was transferred to a new glass tube and mixed with a pre-prepared isolation solution, vortexed for 30 seconds, and incubated at 4° C. for 2. hours. The middle “fluff” layer was transferred onto a PUREEXO® brand column without disturbing the top and bottom layers. The column was spun at 2,000×g for 5 minutes and the cloudy top layer was collected by flow-through.

Electron microscopy examination of isolated exosomes. Isolated exosomes in PBS were fixed in 2% paraformaldehyde (Electron Microscopy Science, Hatfield, Pa., United States of America) in PBS for 2 hours at 22° C. followed by 1% glutaraldehyde (Electron Microscopy Science) for 30 minutes at 22° C. 15 μl of fixed samples were put on a 2% agarose gel with formvar/carbon-coated nickel grids on top and allowed to absorb for 5-10 minutes. The grids with adherent exosomes were fixed in 2% paraformaldehyde in PBS for 10 minutes followed by extensive washing in PBS. Negative contrast staining was performed with 1.9% methyl cellulose and 0.3% uranyl acetate for 10 minutes. The grids with negatively stained exosomes were dried before observation under a Zeiss EM 900 electron microscope.

Size distribution and Zeta potential analysis. Size distributions and Zeta potentials of exosomes were analyzed by a Zetasizer Nano ZS (Malvern Instruments Ltd., Southborough, Mass., United States of America). Briefly, exosomes were washed in ddH2O by centrifugation at 100,000×g for 45 minutes, resuspended with 1 ml ddH2O, and transferred into cuvettes for analysis.

Macrophage depletion. Macrophages were depleted by administration of clodrosomes (Encapsula NanoSciences LLC, Brentwood, Tenn., United States of America; see also PCT International Patent Application Publication No. WO 2017/176792, incorporated by reference in its entirety). Briefly, 150 μl (700 μs) of clodrosomes were intravenously injected into BALB/c mice. The presence of macrophages in mouse liver after the clodrosome treatment was checked by staining with an anti-mouse F4/80 antibody.

Immunofluorescent staining. For Kupffer cell staining, the mice injected with clodrosomes were sacrificed at different time points (24, 48, and 72 hours). Liver tissue was removed and fixed with Periodate-Lysine-Paraformaldehyde (PLP) fixative at 22° C. for 2 hours, dehydrated with 30% sucrose solution at 4° C. overnight, embedded in O.C.T. Compound (Thermo Fisher Scientific, Waltham, Mass., United States of America) and cut into 8 μin sections. The tissue sections were blocked with 5% bovine serum albumin (BSA; Sigma-Aldrich Corp., St. Louis, Mo., United States of America) at 22° C. for 45 minutes, and then incubated with rat anti-mouse F4/80 antibody (1:100) at 37° C. for 2 hours. After three washes, tissue sections were stained with ALEXAFLUOR®-488 or ALEXAFLUOR®-647 conjugated anti-rat secondary antibody (1:800) at 37° C. for 30 minutes and DAN for 90 seconds. The tissue slides were mounted and checked using a confocal microscope equipped with a digital Image analysis system (Pixera, San Diego, Calif., United States of America).

To check the distribution of particles in normal mice with or without clodrosome or exosome injection, PKH67- or PKH26-labeled GNVs (200 nmol) were injected intravenously into mice. Mice were sacrificed 12 hours after the injection. Tissues including liver, lung, and spleen were fixed, dehydrated, and sectioned into 8 μm sections. The tissue sections were stained with DAPI at 22° C. for 90 seconds.

To verify the cell target of nanoparticles in lungs having 4T1 tumor metastasis, 4T1 tumor-bearing mice were treated with 200 μg exosomes and then PKH26-labeled GNVs (200 nmol) were injected intravenously into mice. 12 hours after injection, lung tissue was removed, fixed, dehydrated, and sectioned into 8 μm sections. The tissue sections were blocked with 5% BSA at 22° C. for 45 minutes, incubated with anti-mouse F4/80 at 37° C. for 2 hours, and then stained with ALEXAFLUOR®-labeled secondary antibody at 37° C. for 30 minutes. The co-localization of GNVs with cells was examined by confocal microscope.

Measurement of the concentration of Paclitaxel in mouse tissues. B16F10 melanoma mice were injected intravenously with free paclitaxel or GNV-paclitaxel. Exosome (200 μg) pre-treated B16F10-bearing mice were intravenously administrated GNV-paclitaxel three times, and the paclitaxel in liver, lung, tumor, and spleen tissues was quantitatively analyzed using high performance liquid chromatography (HPLC) as described in Deng et al., 2017. See also U.S. Patent Application Publication No. 2016/0045448 and PCT International Patent Application Publication No. WO 2018/098247, the entire disclosure of each of which is incorporated herein by reference.

B16F10 and 4T1 tumor models. 5×10⁴ B16F10 cells were intravenously injected into six-week-old female C57BL/6 mice. Beginning 5 days later, mice were treated intravenously every 3 days for a total of ten times with PBS, free DTIC/paclitaxel, GNV-DTIC/paclitaxel, or Exo/GNV-DTIC/paclitaxel.

In a second set of experiments, six-week-old female BALB/c mice were injected in a mammary fat pad with murine breast tumor 4T1 cells (1×10⁵ cells/mouse in 50 μl PBS). Beginning 5 days later, mice were treated every 3 days for a total of ten times with GNV-Dox, Exo/GNV-Dox, GNV-miR18a, Exo/GNV-miR18a, GNV-miR18a/Dox, or Exo/GNV-miR18a/Dox, respectively. Growth of tumors was measured and metastasis of tumors in lungs was imaged.

Hematoxylin and Eosin (H&E) staining. Lungs from B16F10 and 4T1 bearing mice were fixed in 2% PLP fixative at 22° C. for 2 hours, dehydrated in 30% sucrose solution overnight at 4° C., embedded in O.C.T. Compound, and sectioned into 8 μm sections. The tissue sections were stained with H&E.

Human blood samples. Anticoagulated blood was collected from volunteer healthy subjects and used for isolation of exosomes with a standard differential centrifugation protocol (see e.g., U.S. Pat. No. 7,897,356; U.S. Patent Application Publication No. 2013/0273544; PCT International Patent Application Publication No. WO 2013/084000; the disclosure of each of which is incorporated herein by reference in its entirety).

Cell culture, transfection and FACS analysis. U937 cells were plated at 9.3×10³ per well in black-wailed 96-well plates (Corning-Costar Inc., Corning, N.Y., United States of America) in antibiotic-free growth medium (Invitrogen) 16 hours before transfection. Transfections were performed using a SIPORT™ brand Amine Transfection Agent (Thermo Fisher Scientific, Waltham, Mass., United States of America) with siRNAs (final concentration 50 nM). Transfections were performed in duplicate and quadruplicate if knockdown was evaluated.

Transfected cells were incubated for 48 hours to allow target knockdown, and then 30 minutes after exosomes isolated from the peripheral blood of healthy subjects were added to each siRNA transfected well. PKH26-labeled GNVs were added for an additional 0-2 hours incubation at 37° C. The treated cells were then washed and PKH26 positive cells were FACS analyzed using a method as described in U.S. Patent Application Publication No. 2014/0308212, the entire disclosure of which is incorporated by reference herein. FlowJo Flow Cytometry Analysis Software (FlowJo, LLC, Ashland, Oreg., United States of America) was used for analysis.

Statistical Analysis. All statistical analyses in this study were performed with SPSS 16.0 software (IBM Corp., Armonk, N.Y., United States of America). Data are presented as mean±SD. The significance of mean values between two groups was analyzed by the Student's t-test. Differences between individual groups were analyzed by one- or two-way analysis of variance test. Differences were considered significant when the p value was less than 0.05, 0.01, or 0.001 as indicated.

### Example 1

### Injection of Exosomes Isolated from Peripheral Blood Enhanced Accumulation of GNVs and Liposomes from the Liver and Spleen to the Lungs

Despite the many potential advantages for using nanoparticles and liposomes as a therapeutic agent delivery system, most of these nanoparticles end up in the liver and spleen, thus delivery of targeted therapeutic agent to the appropriate tissue is prevented and this presents a huge challenge for effective therapy. Exosomes are released from many different types of cells and are continuously circulating in the blood. Although circulating exosomes provide a promising approach to assess biomarkers in human disease, their role(s) in terms of modulating the route of therapeutic nanoparticles injected intravenously is not known.

As such, whether circulating exosomes had an effect on the targeted delivery of therapeutic agents and whether these exosomes could improve the therapeutic efficiency and effectiveness of nanovectors in treatment of various diseases was investigated. It was first determined whether

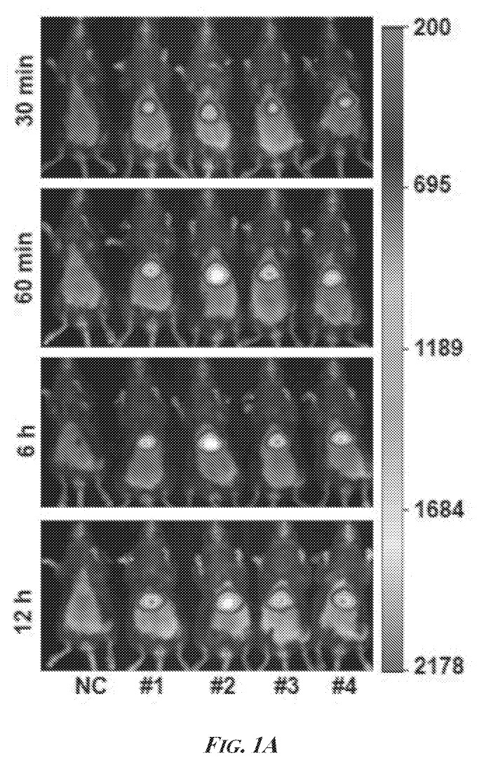

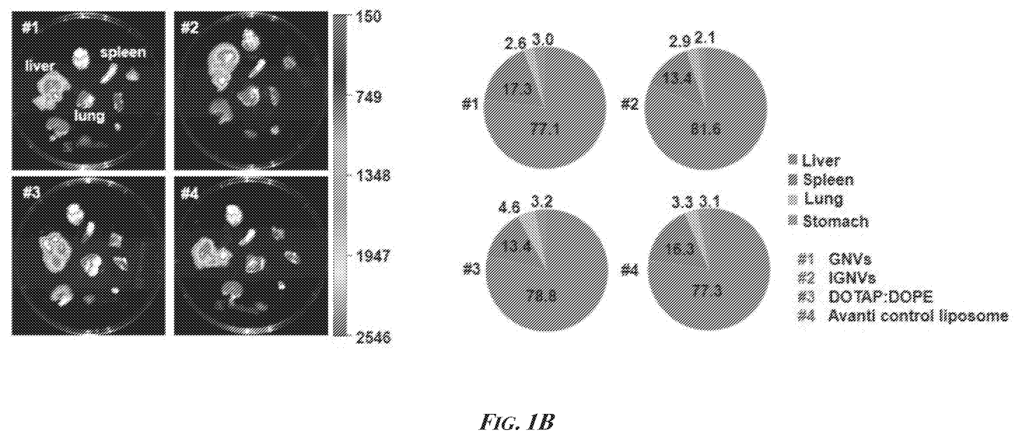

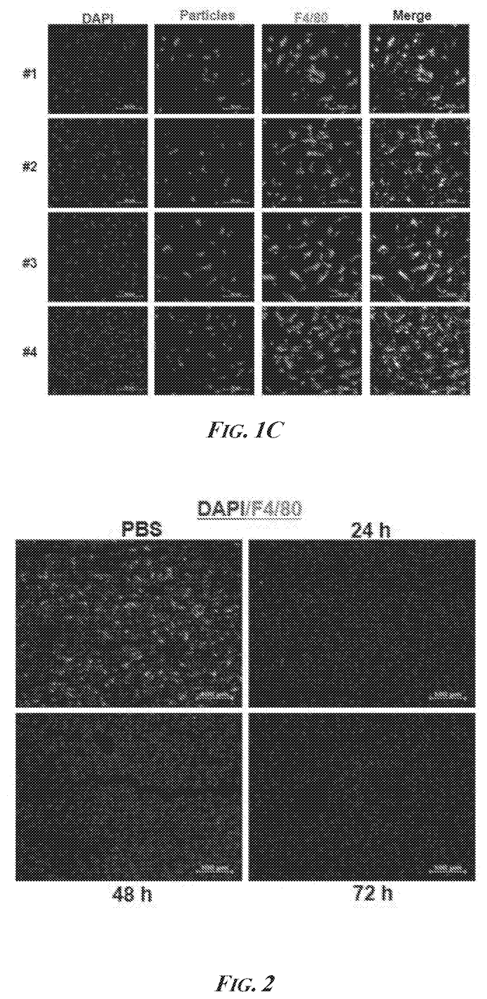

Next, whether circulating exosomes homed to the same tissue as nanovectors that were tested. An in vivo imaging analysis indicated that the majority of nanovectors homed to the liver and spleen within 12 hours after a tail vein injection (FIGS. 1A and 1B). Confocal imaging data further demonstrated that Kupffer cells took up the injected nanoparticles including GNVs and IGNVs as well as commercial liposomes (FIG. 1C).

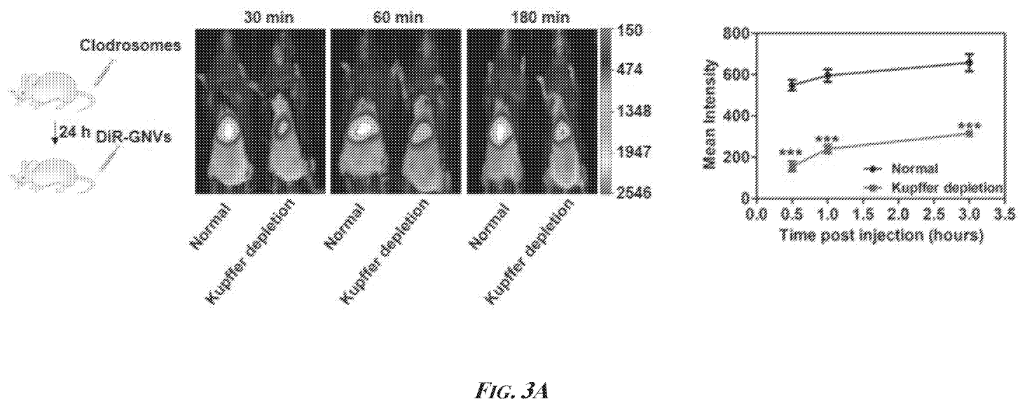

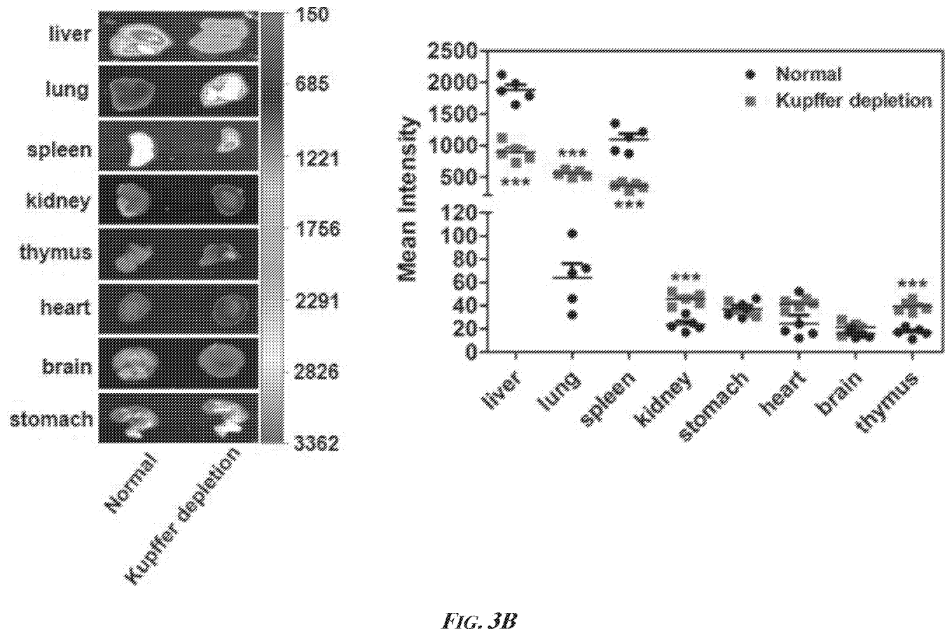

Furthermore, the data generated from depletion of Kupffer cells (FIG. 2) indicated that the depletion of liver Kupffer cells led to a redirection of GNVs from the liver and spleen to the lungs, which correlated with the reduction of GNV signals in the liver and spleen (FIG. 3A-3C). Collectively, these data suggested that i.v. injected nanovectors were taken up by Kupffer cells and depletion of Kupffer cells redirected the nanoparticles to the lungs.

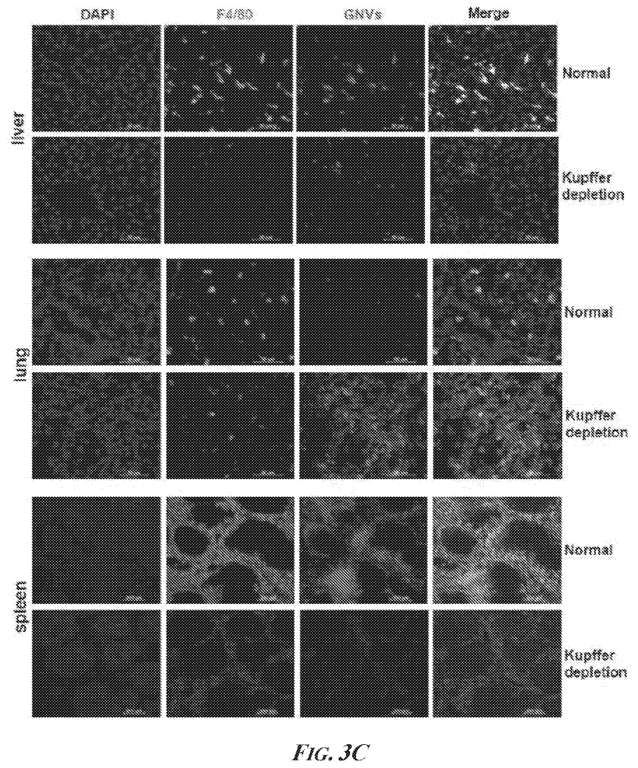

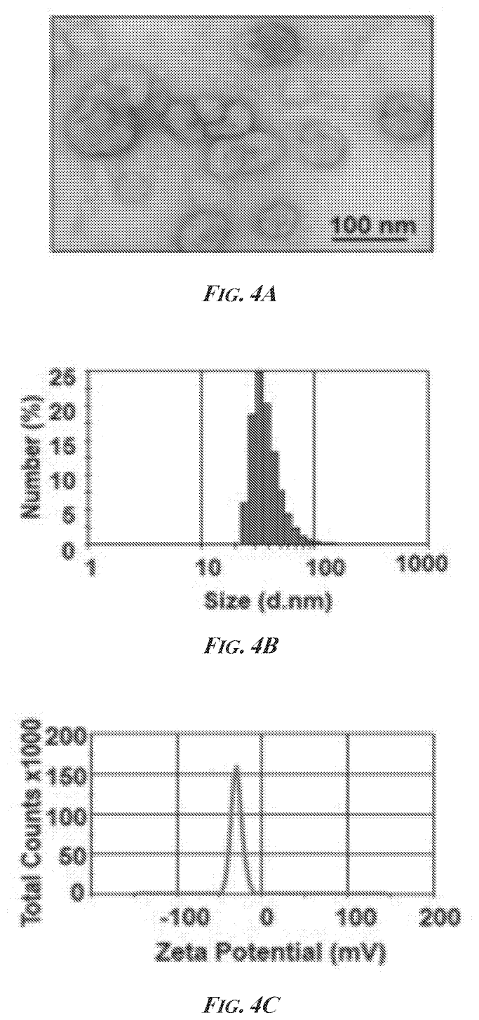

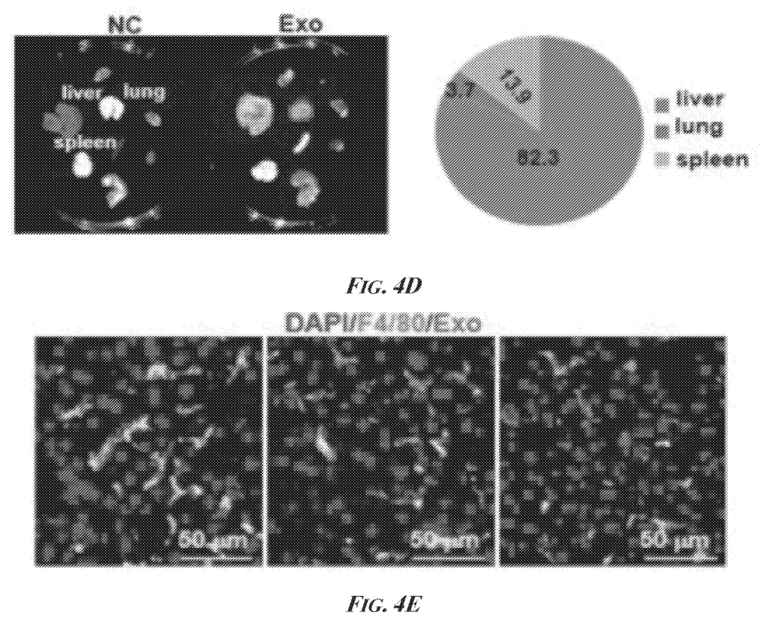

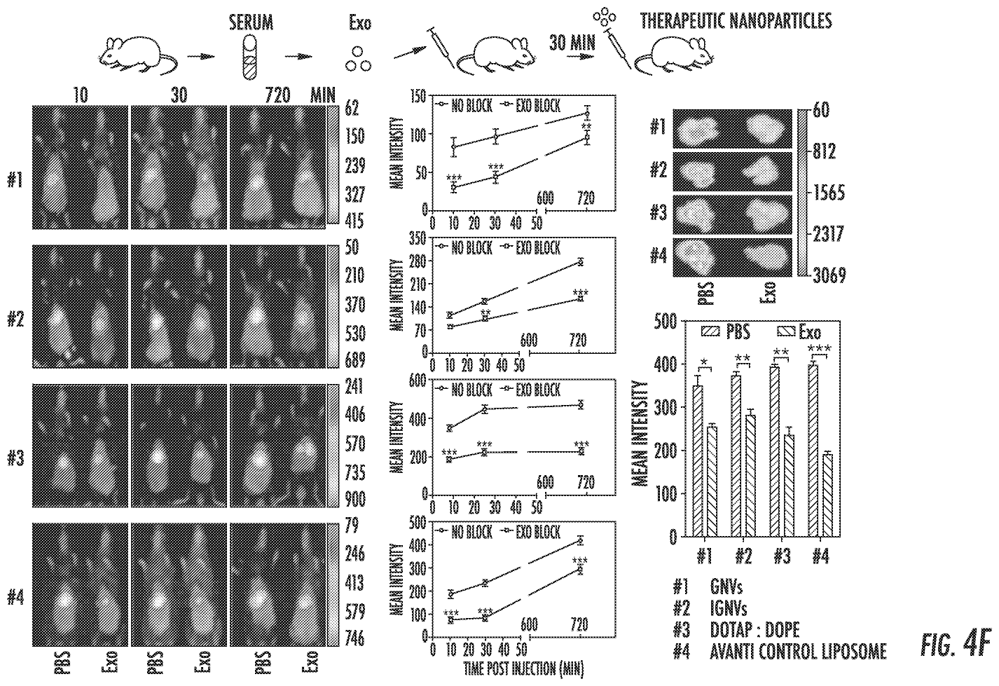

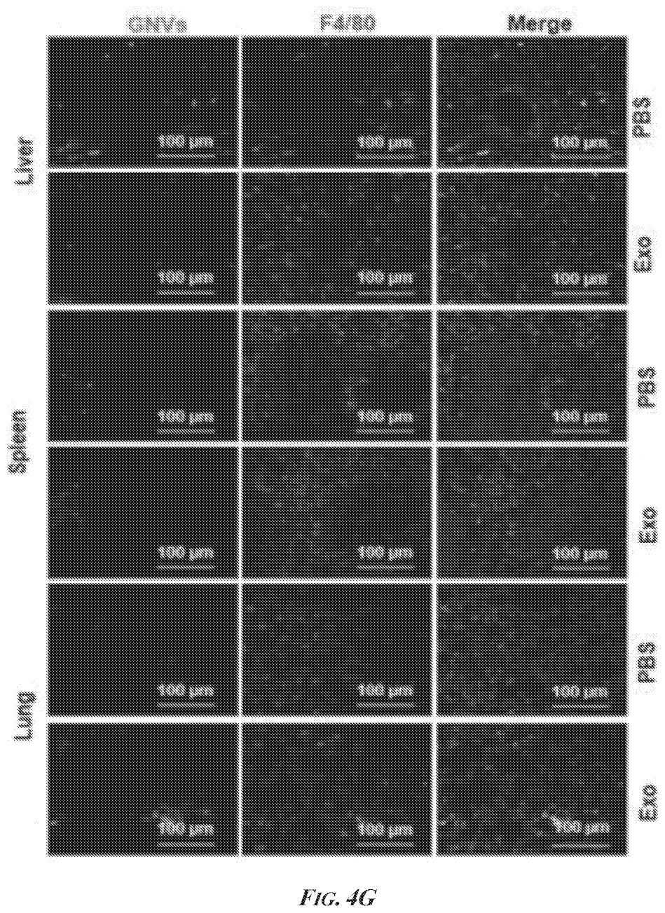

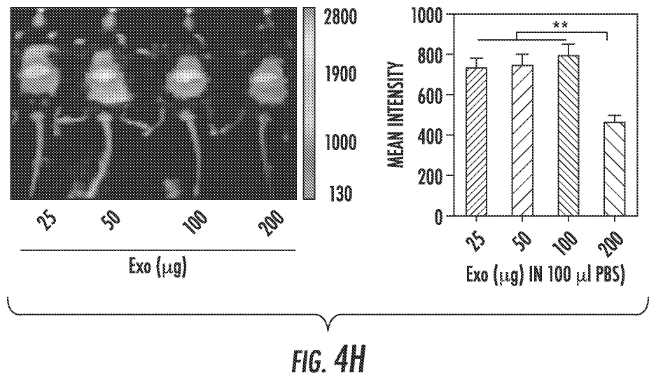

However, from a clinical application standpoint, pre-depletion of a patient's Kupffer cells to prevent liver up take of therapeutic nanovectors would not be acceptable. Therefore, whether injection of autologous exosomes back into the patient before delivery of the therapeutic nanovector would block subsequent liver Kupffer cell uptake was tested. Exosomes isolated from the peripheral blood of mice were examined using electron microscopy imaging (FIG. 4A). In addition, size distributions (FIG. 4B) and Zeta potentials (FIG. 4C) were evaluated, and the results indicated that isolated particles were nano-sized with negative charges. After a tail vein injection, exosomes trafficked to liver (FIG. 4D) and were taken up by Kupffer cells (FIG. 4E). The intensity of the signal evidenced by increasing accumulation of the exosomes increased over a 72-hour period after the injection (FIG. 4E). Unexpectedly, 30 minutes after i.v. injection of exosomes, the intensity of the liver signal of all 4 different types of nanoparticles tested was reduced significantly (FIG. 4F), whereas the number of positive GNVs in the lung was increased (FIG. 4G). The increased signal in the lung of injected GNVs was associated with a decreasing GNV signal in the liver in a dose dependent manner (FIG. 4H). No abnormalities were observed in terms of body weight of mice injected i.v. with autologous exosomes (200 μg in 100 μL PBS) compared with PBS alone (FIG. 4I).

### Example 2

### Injection of Exosomes from Peripheral Blood Enhanced Accumulation of GNVs in Tumors and Lungs

Most cancer deaths result from metastasis. The lungs are the most common clinically relevant sites of cancer metastases including breast cancer, where metastatic breast cancer remains a therapeutic challenge.

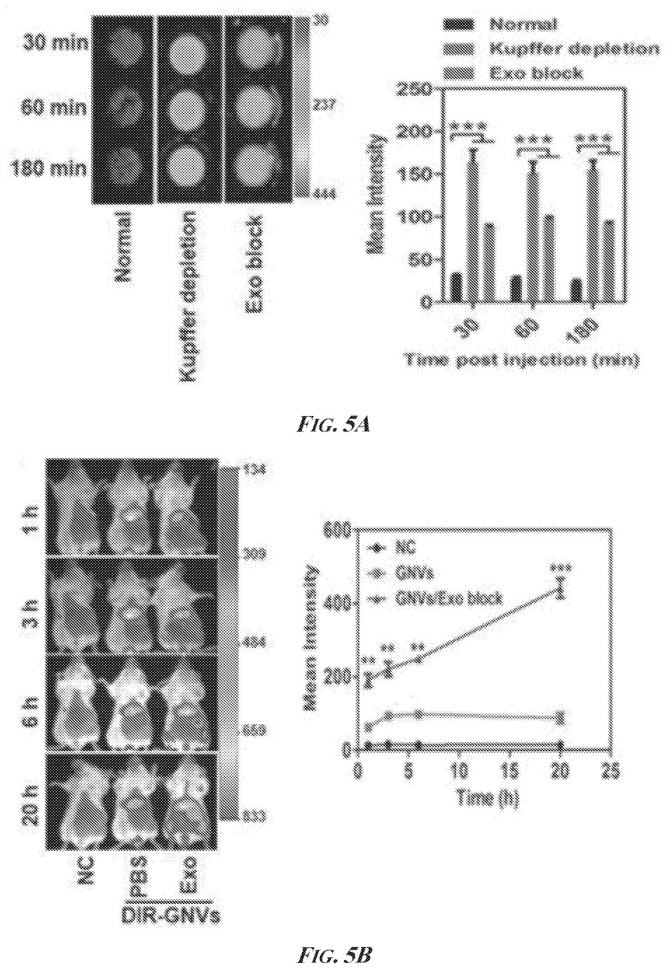

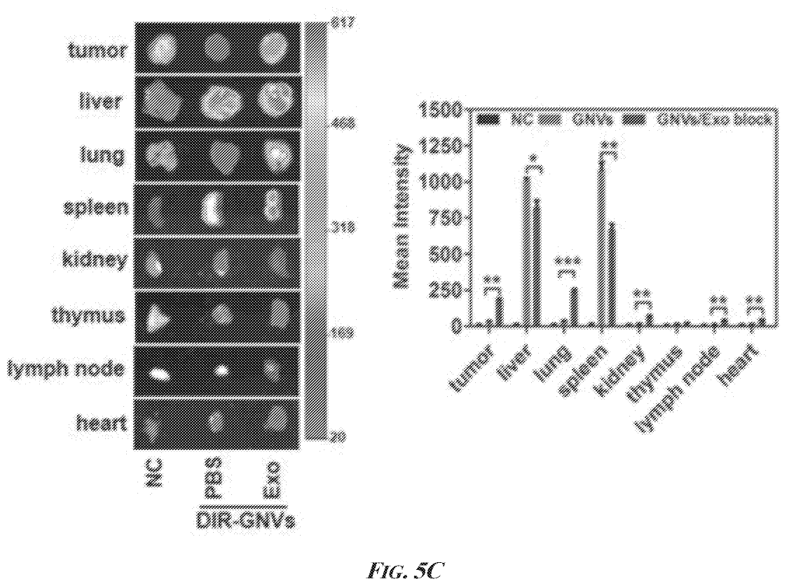

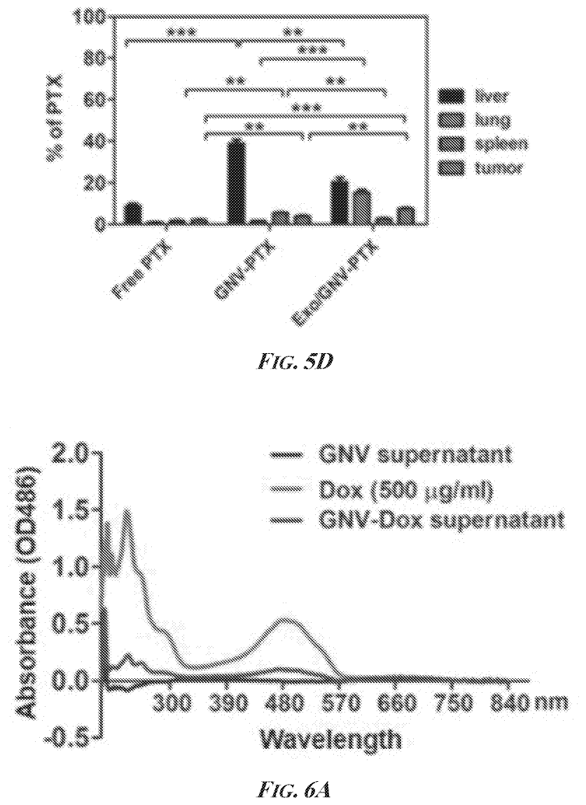

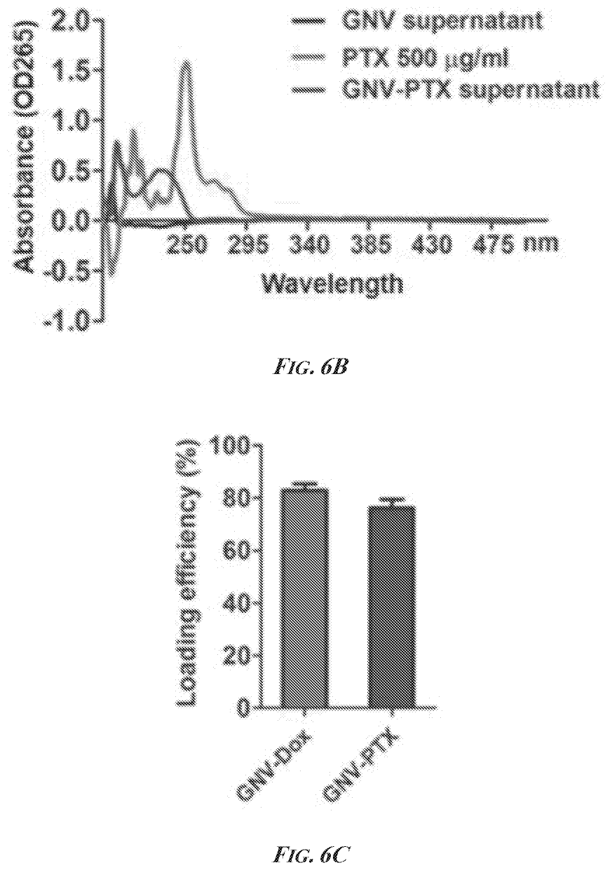

To determine whether injection of previously isolated circulating exosomes leads to enhancing accumulation of GNVs in tumor and lungs, the metastatic mouse mammary carcinoma 4T1 model was used. The mammary pads of mice were injected with 4T1 and at day 14 post-injection, mice were injected i.v. with exosomes purified from circulating blood of BALB/c mice or with PBS as a control. Thirty minutes later, mice were injected iv. with Dir dye-labeled GNVs. Live mouse imaging data indicated that pre-injection of exosomes significantly enhanced the GNV signals detected in circulating blood (FIG. 5A) and breast tumors (FIG. 5B). This result was corroborated by quantitative analysis of the GNVs signals in the tumor and lungs (FIG. 5C) and delivery of the chemotherapeutic drug PTX by GNVs (FIG. 5D). More than 80% of the drugs, including Dox and PTX, were efficiently coupled with GNVs (FIGS. 6A-6C).

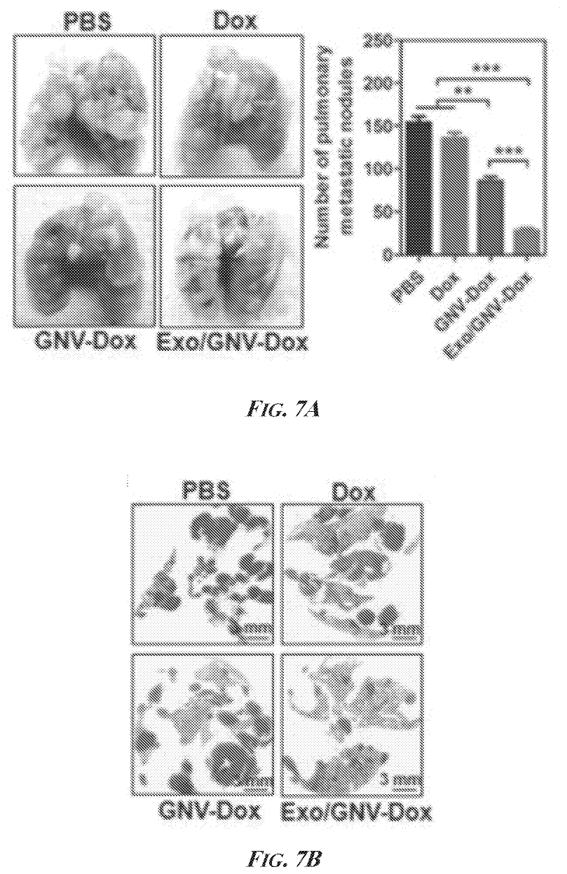

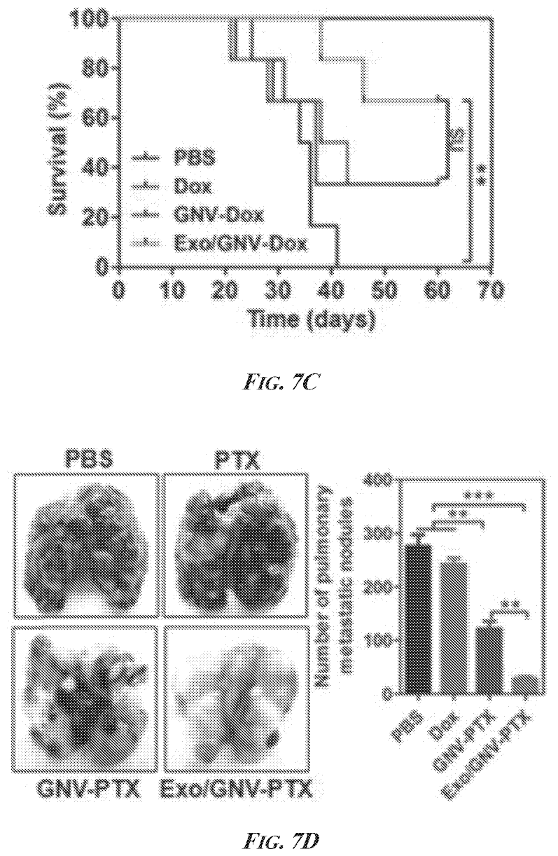

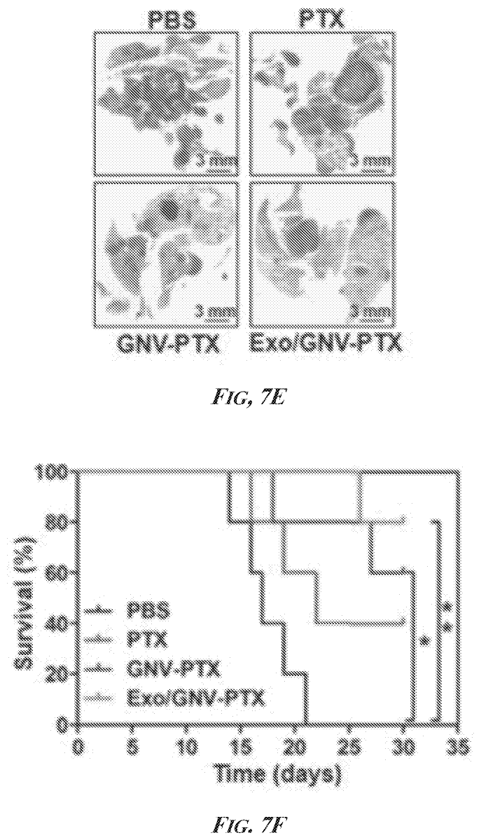

Next, whether observing an enhanced GNV signal in the lung led to better therapeutic effects in regard to inhibiting 4T1 breast tumor metastasis was tested. miR18a, which has anti-cancer immune effects, and the chemotherapeutic drug Dox were co-delivered by GNV. On day 35 post-tumor cell injection, mice with 4T1 tumor cells succumbed to significant lung metastases (FIG. 7A), and all mice (n=10) died by 7 weeks post-injection of tumor cells. In contrast, mice pre-injected with autologous exosomes, followed by i.v. administered GNVs carrying Dox or miR18a or a combination of Dox and miR18a, had a decreased number of macro- (FIG. 7A) and micro- (FIG. 7B) metastatic tumor nodules when compared to mice pre-injected with PBS as a control. Pre-injection with exosomes followed by i.v. administration of GNV carrying Dox significantly prolonged the survival rate of 4T1-bearing mice with lung metastases (FIG. 7C).

To determine whether pre-injection of exosomes isolated from peripheral blood would result in reduced lung metastatic potential, beginning 5 days after tail vein injection of tumor cells, mice were treated with GNV carrying PTX every 3 days for 30 days. Despite the fact that this route bypasses several of the steps occurring during metastasis, it provided an ability to focus on the potential effect of exosomes injected at the final stages of metastasis. Injection of exosomes and GNV-PTX resulted in decreased numbers of macro lung metastases in the mice injected with B16F10 cells. The results indicated that mice preinjected with exosomes followed by i.v.-administered GNV carrying PTX had fewer lung macro- and micro-metastatic tumor nodules than mice that were not injected with exosomes or injected with PBS as a control (FIG. 7D). That the tumors were melanomas in mice pre-injected with exosomes was confirmed by histological analysis of H&E-stained sections (FIG. 7E). This result was also supported by the fact that the lowest mortality was observed in the group of mice pre-injected with exosomes, followed by i.v. administration of GNVs carrying PTX (FIG. 7F).

To test whether the enhanced GNV signal in the lung and breast tumor was applicable to other types of cancer, the murine melanoma B16F10 model was used since the current therapy of lung metastasis for melanoma is disappointing. Therapeutic effects of DTIC/PTX on the prevention of growth of i.v. injected B16F10 tumor cells in the lung was determined.

### Example 3

### CD36- and IGFR1 Receptor-mediated Pathways Played a Role in Exosome-Mediated Prevention of Uptake of GNV Nanoparticles

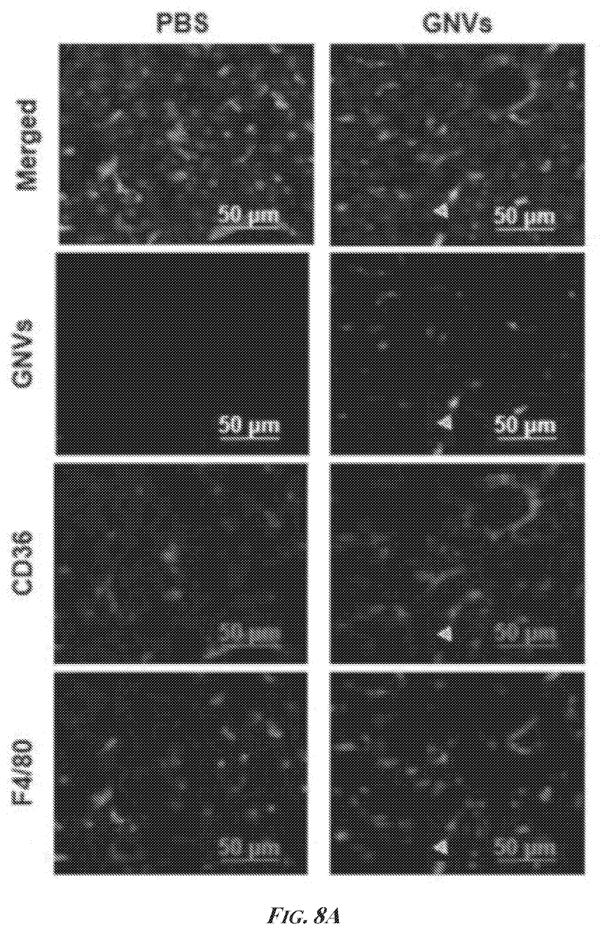

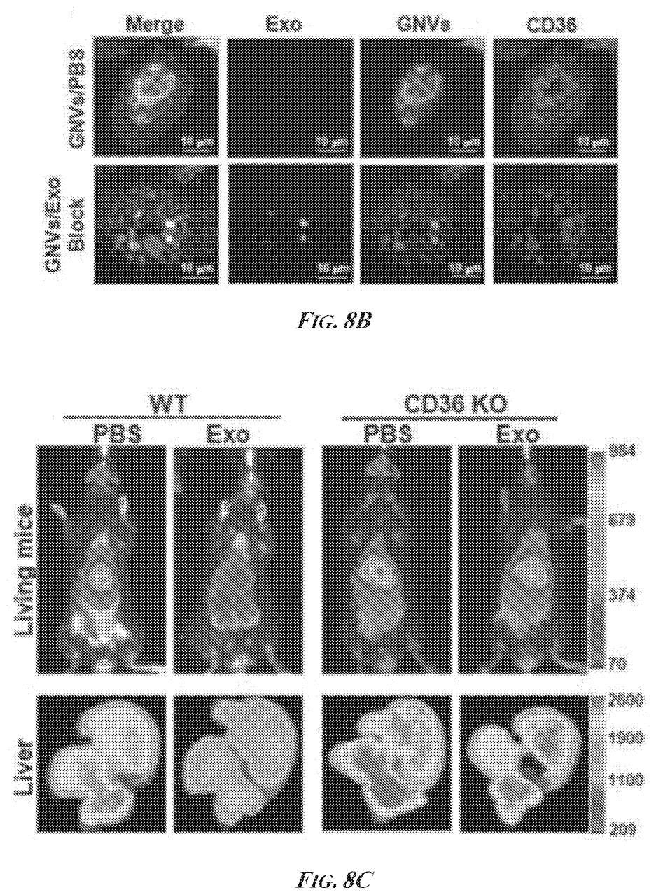

Collectively, the data suggested that exosomes were rapidly sequestered by liver macrophages and prevented subsequent macrophage uptake of GNV nanoparticles. Next, whether the prevention of uptake of GNVs was a macrophage receptor-mediated event was tested. CD36 is known as fatty acid translocase (FAT) and binds many ligands. To determine whether macrophage CD36 plays an in vivo role in exosomes entry, confocal imaging analyses of localization of GNVs with liver macrophages that were F4/80⁺ and CD36⁺ was investigated. The data indicated that F4/80⁺ and CD36⁺ liver macrophages took up GNVs 30 minutes after i.v. injection (FIG. 8A).

To determine whether pre-injection of exosomes had an effect on the location of GNVs in CD36⁺ liver macrophages, mouse liver F4/80⁺ macrophages were isolated from mice pre-injected with exosomes or with PBS as a control. In the control group of mice injected i.v. with PBS, immunohistological staining revealed CD36 was clustered at the outer nuclear membrane and co-localized with GNVs (FIG. 8B, top panel). In contrast, when exosomes were pre-injected, the CD36 cluster at the outer nuclear membrane was not observed, and there was a much weaker GNV signal on the outside of the nucleus (FIG. 8B, bottom panel).

The role of macrophage CD36 in the uptake of GNVs was further demonstrated by in vivo imaging analysis. In wild type B6 mice used as a control, the liver GNV signal was reduced significantly (n=5) when mice were pre-injected with exosomes, compared with the liver signal of mice pre-injected with PBS (FIG. 8C). CD36 knockout (KO) led to reducing the GNV signal intensity difference between mice pre-injected with exosomes and mice pre-injected with PBS (FIG. 8C). These data indicated that uptake of exosomes led to blocking CD36/GNV cluster formation at the outer membrane of the nucleus. The exosome-mediated reduction of cluster formation was associated with the subsequent inhibition of GNV entry into liver macrophages.

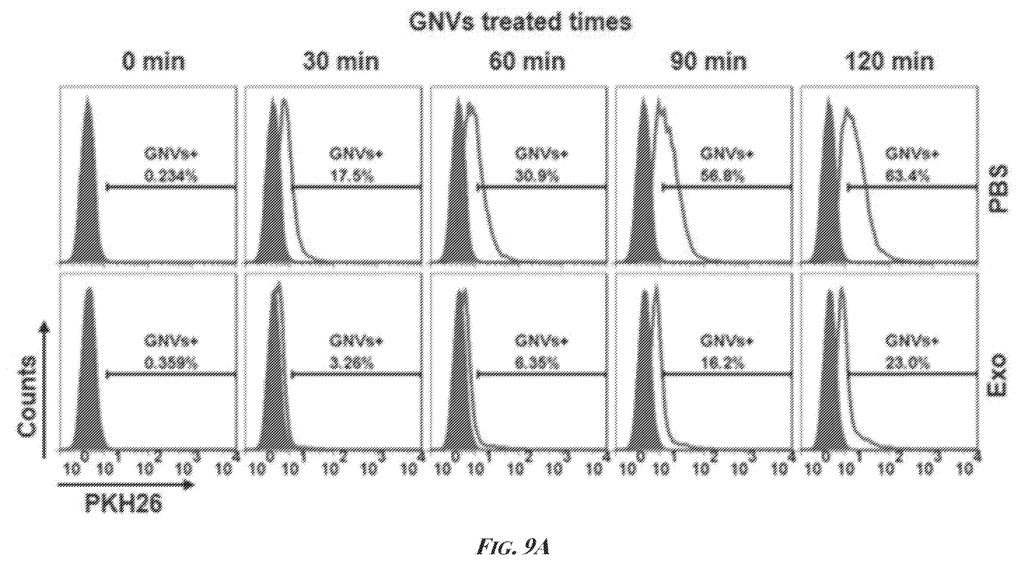

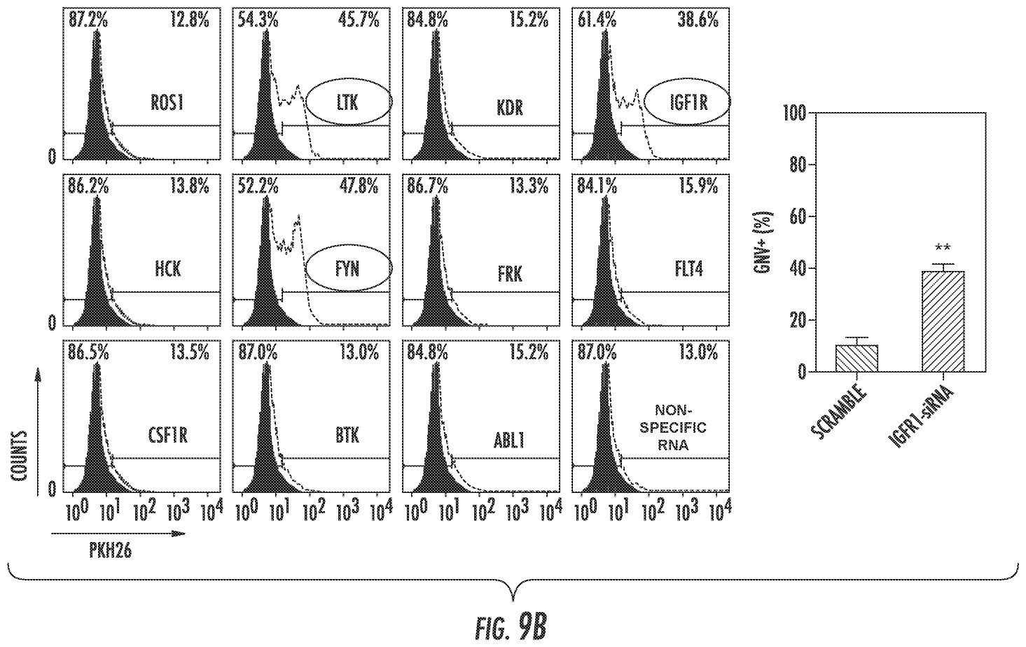

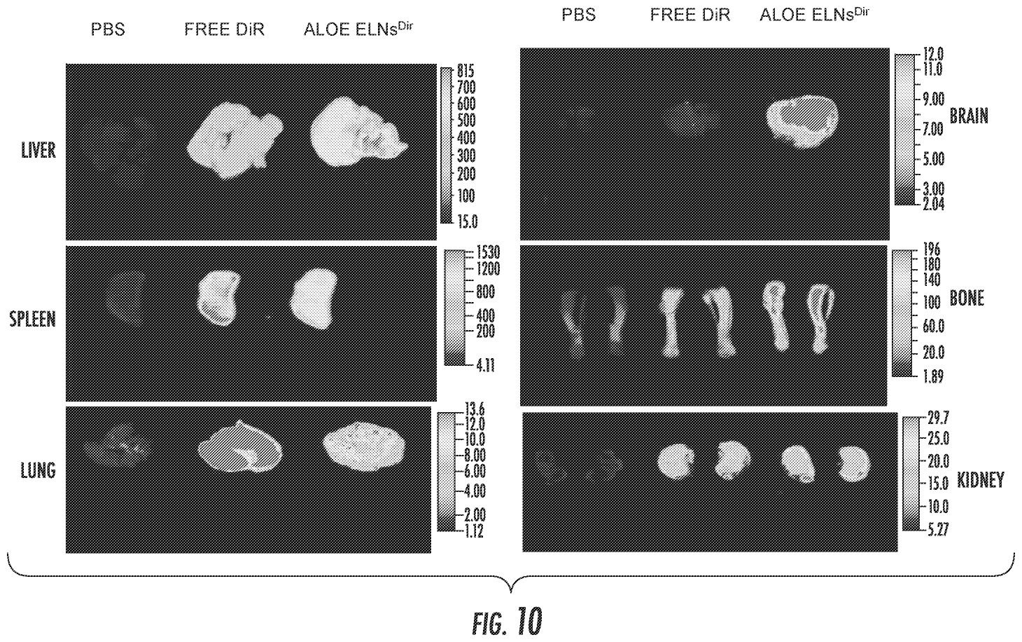

It has been reported that the resulting formation of CD36 clusters initiates signal transduction and internalization of receptor-ligand complexes and tyrosine-family kinases is required for CD36 clustering. How the exosomes regulated kinase(s) that prevents subsequent GNV entry into macrophages is not known. From a clinical application standpoint, human monocytes were used to address this question. FACS analysis of U937 human monocytes pre-treated with or without exosomes isolated from peripheral blood of healthy subjects indicated that exosomes inhibited U937 uptake of GNVs as early as 30 minutes after exosome treatment (FIG. 9A). Using siRNA technology, the results generated from a human tyrosine kinase siRNA library screening assay indicated that knockdown of LTK, IGFR1, and FYN genes completely reversed the exosome-mediated inhibition of the GNV entry effect (FIG. 9B, left panel). The efficiencies of LTK and FYN siRNA knockdown were highly variable, although the cause(s) of the observed variation were not investigated. However, the efficiency of the IGFR1 siRNA knockdown was highly reproducible (FIG. 9B, right panel).

### Example 4

### Aloe ELNs (AELNs) Preferentially Traffic to Brain

To determine the tissue distribution of aloe ELNs (AELNs), 50 mg of DiR fluorescent dye-labeled AELNs were administered to mice orally. 10 days late, mice were sacrificed and DiR fluorescent signals in tissues were detected and measured using an Odyssey Infrared Imager (LI-COR Inc., Lincoln, Nebr., United States of America) as described in Zhuang et al. The results are presented in FIG. 10.

Briefly, after i.v. tail injection, DiR fluorescent signals from AELNs were predominantly detected in liver, brain, and bone, whereas DiR fluorescent signals in mice injected with equal amount of free DiR were predominantly detected in spleen and lung. No visible abnormality was noted in any group of mice.

### Discussion of the Examples

Uptake of circulating exosomes in the liver leads to a reduction in uptake of subsequently administered therapeutic nanovectors via i.v. injection. In some embodiments of the presently disclosed subject matter, it has now been surprisingly found that autologous exosomes are predominately taken up by liver Kupffer cells, and are involved in regulating the distribution of i.v. injected nanovectors. In certain embodiments, increasing the level of peripheral blood-derived exosomes by i.v. injection, blocked accumulation of subsequently injected nanovectors in the liver. Uptake of peripheral blood-derived exosomes led to redirecting grapefruit-derived nanovectors (GNV) i.v. injected from the liver to the lungs under “normal” physiological conditions and to the tumor in breast and melanoma tumor bearing mouse models. The CD36- and IGFR1-mediated pathways play a role in exosome mediated distribution of injected nanovector. It was also found that injection of exosomes isolated from circulating blood enhanced the therapeutic agents delivered by GNV to tumor and prevented lung metastasis. These findings indicated that Kupffer cells play a role in maintaining homeostasis of circulating exosomes and that circulating exosomes are capable of enhancing the efficiency of targeted delivery of therapeutic agents to lungs where most human tumor metastasis occurs. This procedure could be done by isolating exosomes from the peripheral blood of a patient, which are then perfused back into the same patient before the patient is administered therapeutic nanovectors, such as GNVs, as demonstrated in mouse models of this study.

Tumor-specific delivery of therapeutics is challenging. One of the major hurdles for successfully delivering targeted agents by nanovectors is the filtering role of the liver in rapidly sequestering nanovectors in the circulation. Exosomes, i.e., endogenous nanoparticles, are continuously circulating in the peripheral blood and play a role in intercellular communication. Whether the liver sequesters circulating exosomes and whether the level of endogenous exosomes has an effect on the nanovector's delivery efficiency of targeted agents has not been studied. Here, we show that tail vein injected exosomes isolated from mouse peripheral blood are predominately taken up by liver Kupffer cells. Injection of peripheral blood-derived exosomes before i.v. injection of grapefruit-derived nanovector (GNV) decreases the depositing of GNV in the liver and redirects the GNV to the lung and to the tumor in breast and melanoma tumor bearing mouse models. Enhanced therapeutic efficiency of miR18a/Dox or DFTIC/PTX carried by GNVs was demonstrated when there was an injection of exosomes before therapeutic treatment. Furthermore, it was found that CD36 and IGFR1 receptor mediated pathways play a critical role in the exosome mediated inhibitory effect of GNV entry into liver macrophages. Collectively, the findings provide a foundation for using autologous exosomes to enhance therapeutic vector targeted delivery. These findings also provide mechanistic insights into regulation of the blood exosomes homeostasis and its implications for the utilization of autologous exosomes to enhance the efficiency of targeted delivery of therapeutic agents to lungs where most human tumor metastasis takes place.

Thus, disclosed herein are novel biological functions of exosomes and their utility in enhancing targeted delivery of therapeutic agents carried by nanovectors. It has been demonstrated that circulating exosomes were taken up by Kupffer cells, and injection of exosomes into the peripheral blood resulted in a decreased capacity of Kupffer cells to take up subsequently injected GNV nanoparticles and redirect the GNVs from the liver to the lungs. The therapeutic utility of these results was further demonstrated by the inhibition of breast and melanoma lung metastasis in murine models. These findings provide a foundation for further studying the regulatory role of circulating exosomes in terms of response to circulating foreign nanoparticles in general. In addition, this approach has the potential of directly translating into clinical application for treatment of lung related diseases using autologous exosomes.

Furthermore, this study demonstrated that there was a relationship between CD36- and IGFR1-mediated signaling pathways. The findings pointed to the molecular pathway underlying the exosome-mediated inhibition of entry of GNVs into Kupffer cells. The data presented herein showed that this inhibitory effect was CD36- and IGFR1-dependent since knockout of the gene coding for the CD36 receptor or siRNA knockdown of IGFR1 expression negated the inhibitory effect. These observations, together with earlier findings that CD36 can associate with and activate tyrosine-family kinases, provide a defined pathway whereby the receptor triggers internalization of nanovectors like GNVs into Kupffer cells. Both CD36- and IGFR1-mediated pathways could work independently or via crosstalk with each other to control the level of nanoparticles taken up by Kupffer cells. The exosomes circulating in the peripheral blood could serve as an inter-pathway communicator for the crosstalk.

Although the exact nature of the GNV-induced outer nuclear membrane cluster inhibition by exosomes remains to be defined, the fact that the exosome treatment led to blocking the outer nuclear cluster formation induced by GNVs was significant. Proteins gain entry into the nucleus through the nuclear envelope (NE). The NE consists of concentric outer and inner membranes. The NE has important functions in regulating membrane rigidity, gene expression, and chromosome organization. Dysfunctions in NE impair NE architecture and cause human diseases such as rapid aging and cancers. Liposome-like GNVs induce the transient formation of the outer nuclear membrane and endogenous exosomes can inhibit the GNV induced formation of the outer nuclear membrane cluster.

Developing nanovectors for various therapeutic indications, including neurodegenerative disorders, has attracted enormous interest. One of major challenges in this area is to target the delivery of therapeutic agents to the brain and other tissues, where most free drugs cannot enter because of the blood-brain barrier (BBB), blood-retinal barrier, and blood-labyrinth barriers, to name a few. These biological barriers are largely impermeable to drugs, and effective therapeutic drug doses cannot reach the desired pathological sites. Therefore, delivery approaches to meet these challenges are needed.

In addition, most diseases commonly involve large numbers of pathogenic factors that target multiple pathways. Developing an effective therapeutic strategy that can inhibit a plurality of pathogenic factors without causing side effects requires a change from the current approach of delivering individual therapeutic agents, to delivery of a package of therapeutic agents that can target multiple pathogenic factors simultaneously. Delivery vehicles that can selectively target pathogenic sites and carry multiple therapeutic agents without causing toxicity continue to be largely unavailable.

Recently, edible plant-derived exosome-like nanoparticles (ELNs) have been identified, and these consist of a large numbers of lipids, RNA including miRNAs, and proteins. Thus, edible plants including aloe could be beneficial for human health and could be employed to prevent and/or treat diseases including diseases associated with inflammation. Inflammation plays a critical role in a number of brain-, eye-, and ear-related diseases. As set forth herein, it has been shown that edible plant-derived exosomes-like nanoparticles (ELNs) that have anti-inflammatory activities can penetrate the blood-brain barrier, and can be useful for inhibiting a plurality of pathogenic factors without causing side effects. More particularly, after screening a large number of ELNs via intravenous injection in a mouse model, it was determined that aloe ELNs (AELNs) preferentially traffic to brain.

Aloe has been used traditionally as an herbal medicine. It can be taken orally or can be applied to the skin and used for weight loss, diabetes, hepatitis, inflammatory bowel diseases, osteoarthritis, stomach ulcers, asthma, radiation-related skin sores, fever, itching, and inflammation. Thus, the presently disclosed subject matter also provides a foundation for using AELNs as therapeutic agent delivery vehicles, particularly for treatment of brain diseases such as but not limited to inflammatory brain disease where i.v. administration can lead to delivery of therapeutic agents across the blood-brain barrier.

### REFERENCES

All patents, patent applications and patent application publications, scientific journal articles, GENBANK® biosequence database entries, including all annotations therein, biosequences, databases, websites, and other published materials referred to throughout the entire disclosure herein, unless noted otherwise, are incorporated by reference in their entireties.

### Berkow et al. (1997) The Merck Manual of Medical Information, Home ed., Merck Research Laboratories, Whitehouse Station, N.J., United States of America.

### Deng et al. (2017) Molecular Therapy 25:1641-1654.

### Duch et al. (1998) Toxicol Lett, 100-101:255-263.

Ebadi (1998) CRC Desk Reference of Clinical Pharmacology. CRC Press, Boca Raton, Fla., United States of America.

### Freireich et al. (1966) Cancer Chemother Rep 50:219-244.

### Freshney (2016) Culture of Animal Cells: A Manual of Basic Technique and Specialized Applications, John Wiley & Sons, Hoboken, N.J., United States of America.

### Gait (1984) Oligonucleotide Synthesis: A Practical Approach, IRL Press, Oxford, Washington, D.C., United States of America.

### Glover (1985) DNA Cloning, Volumes I and II, IRL Press, Oxford, Washington, D.C., United States of America.

Goodman et al. (1996) Goodman & Gilman's the Pharmacological Basis of Therapeutics, 9th ed. McGraw-Hill Health Professions Division, New York, N.Y., United States of America.

### Hames & Higgins (1984) Transcription and Translation: A Practical Approach, IRL Press, Oxford, Washington, D.C., United States of America.

### Hames & Higgins (1985) Nucleic Acid Hybridisation: A Practical Approach, IRL Press, Oxford, Washington, D.C., United States of America.

### Herzenberg et al. (1996) Weir's Handbook of Experimental Immunology (Four-Volume Set) 5th Ed., Wiley-Blackwell, Hoboken, N.J., United States of America.

IUPAC-IUB Commission on Bio-Chemical Nomenclature Symbols for Amino-Acid Derivatives and Peptides. Recommendations (1971). (1972) Biochemistry 11(9): 1726-1732.
Katzung (2001) Basic & Clinical Pharmacology, 8th ed. Lange Medical Books/McGraw-Hill Medical Pub. Division, New York, N.Y., United States of America

### Mayer & Walker (1987) Immunochemical Methods in Cell and Molecular Biology, Academic Press, London, United Kingdom.

### Miller & Calos (1987) Gene Transfer Vectors for Mammalian Cells, Cold Spring Harbor Laboratory Press, Cold Spring Harbor, N.Y., United States of America.

PCT international Patent Application Publication No. WO 1993/25521; WO 2013/084000; WO 2017/176792; WO 2018/098247.

### Perbal (1984), A Practical Guide to Molecular Cloning, John Wiley & Sons, Hoboken, N.J., United States of America.

Remington et al. (1975) Remington's Pharmaceutical Sciences, 15th ed. Mack Pub. Co., Easton, Pa., United States of America
Sambrook et al. (1989) Molecular Cloning A Laboratory Manual, 2nd Ed. Cold Spring Harbor Laboratory Press, Cold Spring Harbor, N.Y., United States of America; Chapters 16 and 17.
Speight et al, (1997) Avery's Drug Treatment: A Guide to the Properties Choice, Therapeutic Use and Economic Value of Drugs in Disease Management, 4th ed. Adis International, Philadelphia, Pa., United States of America.

### U.S. Patent Application Publication No. 2013/0273544; 2014/0308212; 2016/0045448.

U.S. Pat. Nos. 4,683,195; 5,326,902; 5,234,933; 6,180,082; 7,897,356.

### Woodward (1985) Immobilised Cells and Enzymes: A Practical Approach, IRL Press, Oxford, Washington, D.C., United States of America.

### Wu & Grossman (1987) Methods in Enzymology, Volumes 154 (Recombinant DNA Part E) and 155 (Recombinant DNA Part F). Academic Press Inc., New York, United States of America.

### Zhuang et al. (2015) Journal of Extracellular Vesicles 4:28713.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method for treating a cancer, the method comprising administering to a subject an effective amount of a nanoparticle derived from an edible plant and an effective amount of an autologous exosome.

2. The method of claim 1, wherein the nanoparticle derived from an edible plant comprises, optionally encapsulates, an effective amount of a therapeutic agent.

3. The method of claim 2, wherein the therapeutic agent is a chemotherapeutic agent.

4. The method of any one of the preceding claims, wherein the autologous exosome is to administered at least 30 minutes prior to the administration of a nanoparticle derived from the edible plant.

5. The method of any one of the preceding claims, wherein the cancer is lung cancer.

6. The method of claim 5, wherein the lung cancer is a metastasis in the lung.

7. The method of claim 6, wherein the metastasis is secondary to a melanoma or a breast cancer.

8. A method for enhancing accumulation of a nanoparticle in a lung of a subject, the method comprising administering an effective amount of an autologous exosome to the subject and administrating an effective amount of the nanoparticle to the subject subsequent to the administration of the autologous exosome.

9. The method of claim 8, wherein the nanoparticle comprises, optionally encapsulates, an effective amount of a therapeutic agent.

10. The method of any one of claims 8 and 9, wherein the nanoparticle is derived from an edible plant.

11. A method for delivering an agent to the liver, brain, and/or bone of a subject in need thereof, the method comprising administering to the subject an effective amount of an aloe-derived exosome-like nanoparticle (AELN) comprising, optionally encapsulating, the agent, wherein the administering is via a route of administration such that AELN enters the subject's circulation.

12. The method of claim 11, wherein the agent is a therapeutic agent, optionally a chemotherapeutic agent.

13. The method of one of claims 11 and 12, wherein the subject has a disease, disorder, or condition of the liver, brain, and/or bone at least one symptom and/or consequence of which can be ameliorated by the agent.

14. The method of any one of claims 11-13, wherein the route of administration is intravenous administration.

15. A method for delivering an agent across the blood-brain barrier of a subject, the method comprising administering to the subject an effective amount of an aloe-derived exosome-like nanoparticle (AELN) comprising, optionally encapsulating, the agent via a route of administration wherein AELN enters the subject's circulation resulting in the AELN contacting the blood-brain barrier of the subject, whereby the agent is delivered across the blood-brain barrier of the subject.

16. The method of claim 15, wherein the agent is a therapeutic agent, optionally a to chemotherapeutic agent.

17. The method of one of claims 15 and 16, wherein the subject has a disease, disorder, or condition of the brain at least one symptom and/or consequence of which is treatable with the agent.
